(19) United States
(12) Patent Application Publication
(10) Pub. No.: US 2019/0307732 A1
(43) Pub. Date: Oct. 10, 2019

(54) METHODS OF TREATING MITOCHONDRIAL AND METABOLIC DISORDERS

(71) Applicant: Abraxis Bioscience, LLC, Summit, NJ (US)

(72) Inventor: Neil P. DESAI, Pacific Palisades, CA (US)

(21) Appl. No.: 16/336,390

(22) PCT Filed: Sep. 28, 2017
(86) PCT No.: PCT/US17/54149

§ 371 Date: Mar. 25, 2019

Related U.S. Application Data
(60) Provisional application No. 62/401,092, filed on Sep. 28, 2016.

(51) Int. Cl. A61K 31/436; A61K 47/42; A61K 9/00; A61K 47/69; A61K 9/51

(52) U.S. Cl. CPC A61K 31/436; A61K 47/42; B82Y 5/00; A61K 47/6929; A61K 9/5169; A61K 9/0019

(57) ABSTRACT

The present invention relates to methods and compositions for the treatment of diseases, such as mitochondrial-associated disorders, for example Leigh, MELAS, and NARP syndrome, and metabolic disorders, comprising administering an allosteric mTOR inhibitor, such as a composition comprising nanoparticles comprising an allosteric mTOR inhibitor and an albumin. Also provided are medicine and kits useful for the methods described herein.

### CROSS-REFERENCE TO RELATED APPLICATION

This application claims priority to U.S. Provisional Application No. 62/401,092, filed on Sep. 28, 2016, which is hereby incorporated by reference in its entirety.

### TECHNICAL FIELD

The present invention relates to methods and compositions for the treatment of diseases, such as mitochondrial-associated disorders, for example Leigh, MELAS, and NARP syndrome, and metabolic disorders, comprising administering an allosteric mTOR inhibitor, such as a composition comprising nanoparticles comprising an allosteric mTOR inhibitor and an albumin.

### BACKGROUND

The mitochondrion is an organelle present in most eukaryotic cells. In addition to generating cellular ATP, mitochondria are also involved in other cellular functions, such as cellular homeostasis, signaling pathways, and steroid synthesis.

Dysfunction of proper mitochondrial activity is linked to numerous mitochondrial-associated disorders. Approximately one in 4,000 children born in the United States every year will develop a mitochondrial-associated disorder by age 10. In adults, many aging disorders are linked to defects in mitochondrial function. Generally, mitochondrial-associated disorders are inherited disorders obtained through, for example, autosomal inheritance, mitochondrial DNA inheritance, and combinations thereof. Mitochondrial-associated disorders can also be caused by, for example, somatic mutations and exposure to mitochondrial toxins.

Generally, treatment of mitochondrial-associated disorders is palliative and aimed at treating mitochondrial-associated disorder symptoms and improving quality of life. Palliative treatments include, for example, administering vitamins, conserving energy, controlling dietary intake, and reducing stress on the body.

The disclosures of all publications, patents, patent applications and published patent applications referred to herein are hereby incorporated herein by reference in their entirety.

### BRIEF SUMMARY OF THE INVENTION

The present application in some embodiments provides a method of treating an individual having a mitochondrial-associated disorder comprising administering to the individual an effective amount of an allosteric mTOR inhibitor. In some embodiments, the individual having a mitochondrial-associated disorder has one or more of the following: an ataxia, a kidney disorder, a liver disorder, a metabolic disorder, a myopathy, a neuropathy, a myelopathy, an encephalopathy, or an oxidative phosphorylation disorder. In some embodiments, the individual having a mitochondrial-associated disorder has Leigh syndrome. In some embodiments. Leigh syndrome is maternally inherited Leigh syndrome. In some embodiments, Leigh syndrome is infantile onset Leigh syndrome, juvenile onset Leigh syndrome, or adult onset Leigh syndrome. In some embodiments, the individual having a mitochondrial-associated disorder has MELAS syndrome. In some embodiments, the individual having a mitochondrial-associated disorder has NARP syndrome. In some embodiments, the individual having a mitochondrial-associated disorder has one or more of the following: an aging disorder, an autism spectrum disorder, a chronic inflammatory disorder, diabetes mellitus, or a fatty acid oxidation disorder.

In some embodiments according to any of the methods described above, the individual having a mitochondrial-associated disorder has a mitochondrial DNA mutation-associated disorder.

In some embodiments according to any of the methods described above, the individual having a mitochondrial-associated disorder has a nuclear DNA mutation-associated disorder.

In some embodiments according to any of the methods described above, the individual having a mitochondrial-associated disorder has an X chromosome mutation-associated disorder.

In some embodiments according to any of the methods described above, the individual is about one month old to about thirty years old.

In some embodiments according to any of the methods described above, the individual is between the ages of about three months old and about two years old at the age of onset.

In some embodiments according to any of the methods described above, the age of onset of one or more mitochondrial-associated disorder symptoms in the individual is between about three months old and about two years old.

In some embodiments according to any of the methods described above, the individual is a male.

In some embodiments according to any of the methods described above, the individual has a mutation in one or more of the following genes: LRPPRC, MT-ATP6, MT-ND1, MT-ND2, MT-ND3, MT-ND5, MT-ND6, MT-TL1, MT-TH, MT-TV, NDUFS1, NDUFS2, NDUFS3, NDUFS4, NDUFS7, NDUFS8, or SURF1.

In some embodiments according to any of the methods described above, the individual is selected for treatment based on the ratio of lactate to pyruvate in the blood, plasma, cerebrospinal fluid, or urine. In some embodiments, the ratio of lactate to pyruvate is at least 10. In some embodiments, the ratio of lactate to pyruvate is at least 20.

In some embodiments according to any of the methods described above, the individual is selected for treatment based on the ratio of lactate to pyruvate in their blood, plasma, cerebrospinal fluid, or urine. In some embodiments, the ratio of lactate to pyruvate is at least 10:1. In some embodiments, the ratio of lactate to pyruvate is at least 20:1.

The present application in some embodiments further provides a method of inhibiting cellular glucose consumption in an individual comprising administering to the individual an effective amount of an allosteric mTOR inhibitor.

The present application in some embodiments further provides a method of reducing cellular glucose consumption in an individual comprising administering to the individual an effective amount of an allosteric mTOR inhibitor. In some embodiments, the individual is characterized by abnormally high cellular glucose consumption in one or more tissues.

The present application in some embodiments further provides a method of treating an individual having a metabolic disorder comprising administering to the individual an effective amount of an allosteric mTOR inhibitor.

The present application in some embodiments further provides a method of treating an individual having a disease comprising administering to the individual an effective amount of an allosteric mTOR inhibitor, wherein the disease is selected from the group consisting of fetal dilated cardiomyopathy, tuberous sclerosis complex (TSC) and related disorders, childhood onset cardiomyopathy. Noonan syndrome, polycystic kidney disease, age-related and genetically induced hypertrophic cardiomyopathy, and a rheumatic disease.

In some embodiments according to any of the methods described above, the allosteric mTOR inhibitor is a composition comprising nanoparticles comprising an allosteric mTOR inhibitor and an albumin. In some embodiments according to any of the methods described above, the allosteric mTOR inhibitor is in a composition comprising nanoparticles comprising the allosteric mTOR inhibitor and an albumin. In some embodiments, the nanoparticles in the composition have an average diameter of no greater than about 150 nm. In some embodiments, the nanoparticles in the composition have an average diameter of no greater than about 120 nm. In some embodiments, the allosteric mTOR inhibitor in the nanoparticles is associated with the albumin. In some embodiments, the allosteric mTOR inhibitor in the nanoparticles is coated with the albumin. In some embodiments, the weight ratio of albumin and allosteric mTOR inhibitor in the nanoparticle composition is about 1:1 to about 9:1. In some embodiments, the weight ratio of albumin and allosteric mTOR inhibitor in the nanoparticle composition is about 1:1 to about 10:1. In some embodiments, the weight ratio of albumin and allosteric mTOR inhibitor in the nanoparticle composition is about 1:1 to about 11:1. In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the albumin is human albumin. In some embodiments, the albumin is human serum albumin.

In some embodiments according to any of the methods described above, the allosteric mTOR inhibitor is a limus drug. In some embodiments, the limus drug is sirolimus.

In some embodiments according to any of the methods described above, the effective amount of allosteric mTOR inhibitor is about 1 mg/m² to about 150 mg/m². In some embodiments according to any of the methods described above, the effective amount of allosteric mTOR inhibitor is about 0.1 mg/m² to about 150 mg/m² (such as about 0.5 mg/m² to about 100 mg/m², or about 1 mg/m² to about 30 mg/m²). In some embodiments, the effective amount of allosteric mTOR inhibitor is administered weekly. In some embodiments, the effective amount of allosteric mTOR inhibitor is administered once every two weeks. In some embodiments, the effective amount of allosteric mTOR inhibitor is administered once every three weeks. In some embodiments, the effective amount of allosteric mTOR inhibitor is administered once every four weeks. In some embodiments, the effective amount of allosteric mTOR inhibitor is administered daily. In some embodiments, the effective amount of allosteric mTOR inhibitor is administered once every three days.

In some embodiments according to any of the methods described above, the effective amount of allosteric mTOR inhibitor is administered intravenously, intraarterially, intraperitoneally, intravesicularly, subcutaneously, intrathecally, intrapulmonarily, intramuscularly, intratracheally, intraocularly, transdermally, intradermally, orally, intraportally, intrahepatically, by hepatic arterial infusion, or by inhalation. In some embodiments, the effective amount of allosteric mTOR inhibitor is administered intravenously.

In some embodiments according to any of the methods described above, the individual is human.

In some embodiments according to any of the methods described above, the individual has not been previously treated with an allosteric mTOR inhibitor.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF THE FIGURES

FIG. 1 shows dose response curves of IMR90 fibroblasts following administration with either nab-sirolimus (also referred to as ABI-009), rapamycin, or torin 1.

FIG. 2 shows the presence (or lack thereof) of pS6, total S6, and vimentin in IMR90 fibroblasts following administration of nab-sirolimus (also referred to as ABI-009) and rapamycin at varying doses.

### DETAILED DESCRIPTION OF THE INVENTION

The present invention provides methods and compositions for treating an individual having a disease, such as a mitochondrial-associated disorder, for example Leigh syndrome, MELAS syndrome, or NARP syndrome, and metabolic disorders, comprising administering to the individual an effective amount of an allosteric mTOR inhibitor, such as an effective amount of a composition comprising nanoparticles comprising an allosteric mTOR inhibitor and an albumin (hereinafter also referred to as “mTOR nanoparticle composition”). In some embodiments, the allosteric mTOR inhibitor is a limus drug. In some embodiments, the limus drug is in a composition comprising the limus drug and an albumin (hereinafter also referred to as “limus nanoparticle composition”). In some embodiments, the allosteric mTOR inhibitor is sirolimus. In some embodiments, the sirolimus is in a composition comprising nanoparticles comprising sirolimus and an albumin. In some embodiments, the albumin is human albumin (such as human serum albumin). In some embodiments, the nanoparticles comprise sirolimus associated (e.g., coated) with albumin. In some embodiments, the average particle size of the nanoparticles in a nanoparticle composition is no more than about 150 nm (such as no greater than about 120 nm). In some embodiments, the sirolimus is in a composition comprising an albumin stabilized nanoparticle formulation of sirolimus. In some embodiments, the allosteric mTOR inhibitor is nab-sirolimus.

In some embodiments, there is provided a method of treating an individual having a mitochondrial-associated disorder, comprising administering to the individual an effective amount of a composition comprising nanoparticles comprising a limus drug and an albumin, wherein the limus drug is associated (e.g., coated) with the albumin. In some embodiments, there is provided a method of treating an individual having a mitochondrial-associated disorder, comprising administering to the individual an effective amount of a composition comprising nanoparticles comprising a limus drug and an albumin, wherein the average particle size of the nanoparticles in a nanoparticle composition is no greater than about 150 nm (such as less than about 120 nm). In some embodiments, there is provided a method of treating an individual having a mitochondrial-associated disorder, comprising administering to the individual an effective amount of a composition comprising nanoparticles comprising a limus drug and an albumin, wherein the limus drug is coated with the albumin, and wherein the average particle size of the nanoparticles in the nanoparticle composition is no greater than about 150 nm (such as no greater than about 120 nm).

In some embodiments, there is provided a method of treating an individual having a mitochondrial-associated disorder, comprising administering to the individual an effective amount of a composition comprising nanoparticles comprising sirolimus and an albumin. wherein sirolimus is associated (e.g., coated) with the albumin. In some embodiments, there is provided a method of treating an individual having a mitochondrial-associated disorder, comprising administering to the individual an effective amount of a composition comprising nanoparticles comprising sirolimus and an albumin, wherein the average particle size of the nanoparticles in the nanoparticle composition is no greater than about 150 nm (such as less than about 120 nm). In some embodiments, there is provided a method of treating an individual having a mitochondrial-associated disorder, comprising administering to the individual an effective amount of a composition comprising nanoparticles comprising sirolimus and an albumin, wherein sirolimus is coated with the albumin, and wherein the average particle size of the nanoparticles in the nanoparticle composition is no greater than about 150 nm (such as no greater than about 120 nm).

In some embodiments, there is provided a method of treating an individual having a mitochondrial-associated disorder, comprising administering to the individual an effective amount of a composition comprising nab-sirolimus.

In some embodiments, there is provided a method of treating an individual having a metabolic disorder, comprising administering to the individual an effective amount of a composition comprising nanoparticles comprising a limus drug and an albumin, wherein the limus drug is associated (e.g., coated) with the albumin. In some embodiments, there is provided a method of treating an individual having a metabolic disorder, comprising administering to the individual an effective amount of a composition comprising nanoparticles comprising a limus drug and an albumin, wherein the average particle size of the nanoparticles in a nanoparticle composition is no greater than about 150 nm (such as less than about 120 nm). In some embodiments, there is provided a method of treating an individual having a metabolic disorder, comprising administering to the individual an effective amount of a composition comprising nanoparticles comprising a limus drug and an albumin, wherein the limus drug is coated with the albumin, and wherein the average particle size of the nanoparticles in the nanoparticle composition is no greater than about 150 nm (such as no greater than about 120 nm).

In some embodiments, there is provided a method of treating an individual having a metabolic disorder, comprising administering to the individual an effective amount of a composition comprising nanoparticles comprising sirolimus and an albumin, wherein sirolimus is associated (e.g., coated) with the albumin. In some embodiments, there is provided a method of treating an individual having a metabolic disorder, comprising administering to the individual an effective amount of a composition comprising nanoparticles comprising sirolimus and an albumin, wherein the average particle size of the nanoparticles in the nanoparticle composition is no greater than about 150 nm (such as less than about 120 nm). In some embodiments, there is provided a method of treating an individual having a metabolic disorder, comprising administering to the individual an effective amount of a composition comprising nanoparticles comprising sirolimus and an albumin, wherein sirolimus is coated with the albumin, and wherein the average particle size of the nanoparticles in the nanoparticle composition is no greater than about 150 nm (such as no greater than about 120 nm).

In some embodiments, there is provided a method of treating an individual having a metabolic disorder, comprising administering to the individual an effective amount of a composition comprising nab-sirolimus.

In some embodiments, the allosteric mTOR inhibitor is administered intravenously. In some embodiments, the allosteric mTOR inhibitor is administered intraportally. In some embodiments, the allosteric mTOR inhibitor is administered intraarterially. In some embodiments, the allosteric mTOR inhibitor is administered intraperitoneally. In some embodiments, the allosteric mTOR inhibitor is administered intrahepatically. In some embodiments, the allosteric mTOR inhibitor is administered by hepatic arterial infusion. In some embodiments, the allosteric mTOR inhibitor is administered intravesicularly. In some embodiments, the allosteric mTOR inhibitor is administered subcutaneously. In some embodiments, the allosteric mTOR inhibitor is administered intrathecally. In some embodiments, the allosteric mTOR inhibitor is administered intrapulmonarily. In some embodiments, the allosteric mTOR inhibitor is administered intramuscularly. In some embodiments, the allosteric mTOR inhibitor is administered intratracheally. In some embodiments, the allosteric mTOR inhibitor is administered intraocularly. In some embodiments, the allosteric mTOR inhibitor is administered transdermally. In some embodiments, the allosteric mTOR inhibitor is administered intradermally. In some embodiments, the allosteric mTOR inhibitor is administered orally. In some embodiments, the allosteric mTOR inhibitor is administered by inhalation.

Individuals having a mitochondrial-associated disorder can be treated with the methods described herein including, but not limited to, individuals having an ataxia, a kidney disorder, a liver disorder, a metabolic disorder, a myopathy, a neuropathy, a myelopathy, an encephalopathy, an oxidative phosphorylation disorder, an aging disorder, an autism spectrum disorder, a chronic inflammatory disorder, diabetes mellitus, and a fatty acid oxidation disorder. In some embodiments, the individual having a mitochondrial-associated disorder has a mitochondrial DNA mutation-associated disorder. In some embodiments, the individual having a mitochondrial-associated disorder has an X chromosome mutation-associated disorder. In some embodiments, the individual having a mitochondrial-associated disorder has a nuclear DNA mutation-associated disorder. In some embodiments, the individual having a mitochondrial-associated disorder has Leigh syndrome, such as maternally inherited Leigh syndrome. In some embodiments, Leigh syndrome is infantile onset Leigh syndrome, juvenile onset Leigh syndrome, or adult onset Leigh syndrome. In some embodiments, the individual having a mitochondrial-associated disorder has MELAS syndrome. In some embodiments, the individual having a mitochondrial-associated disorder has NARP syndrome.

Individuals having a metabolic disorder can be treated with the methods described herein including, but not limited to, disorders associated with cellular glucose consumption (e.g., abnormally high cellular glucose consumption in one or more tissues), disorders associated with insulin resistance, hypoglycemia, hyperinsulinemic hypoglycemia, diabetes mellitus type 1, diabetes mellitus type 2, and metabolic syndrome.

The methods described herein can be used for any one or more of the following purposes: alleviating one or more symptoms in an individual having a mitochondrial-associated disorder, reducing one or more symptoms in an individual having a mitochondrial-associated disorder, preventing one or more symptoms in an individual having a mitochondrial-associated disorder, treating one or more symptoms in an individual having a mitochondrial-associated disorder, ameliorating one or more symptoms in an individual having a mitochondrial-associated disorder, and delaying onset of one or more symptoms in an individual having a mitochondrial-associated disorder.

The methods described herein can be used for any one or more of the following purposes: alleviating one or more symptoms in an individual having a metabolic disorder, reducing one or more symptoms in an individual having a metabolic disorder, preventing one or more symptoms in an individual having a metabolic disorder, treating one or more symptoms in an individual having a metabolic disorder, ameliorating one or more symptoms in an individual having a metabolic disorder, and delaying onset of one or more symptoms in an individual having a metabolic disorder.

Also provided are compositions (such as pharmaceutical compositions), medicine, kits, and unit dosages useful for the methods described herein.

Further provided are methods of treating an individual having a mitochondrial-associated disorder according to any one of the methods described above, wherein the treatment is based on activity level of a biomarker in the individual including, but not limited to, coenzyme Q10 activity, cytochrome oxidase activity, NADH dehydrogenase activity, succinate dehydrogenase activity, complex I activity, complex II activity, complex III activity, complex IV activity, complex V activity, complex I and III activity, complex II and III activity, citrate synthase activity, pyruvate dehydrogenase complex activity, tricarboxylic acid cycle enzymatic activity, and beta-oxidation enzymatic activity. In some embodiments, the methods further comprise determining activity level of a biomarker in the individual including, but not limited to, coenzyme Q10 activity, cytochrome oxidase activity. NADH dehydrogenase activity, succinate dehydrogenase activity, complex I activity, complex II activity, complex III activity, complex IV activity, complex V activity, complex I and III activity, complex II and III activity, citrate synthase activity, pyruvate dehydrogenase complex activity, tricarboxylic acid cycle enzymatic activity, and beta-oxidation enzymatic activity. In some embodiments, the methods further comprise selecting the individual for treatment based on activity level of a biomarker in the individual including, but not limited to, coenzyme Q10 activity, cytochrome oxidase activity, NADH dehydrogenase activity, succinate dehydrogenase activity, complex I activity, complex II activity, complex III activity, complex IV activity, complex V activity, complex I and III activity, complex II and III activity, citrate synthase activity, pyruvate dehydrogenase complex activity, tricarboxylic acid cycle enzymatic activity, and beta-oxidation enzymatic activity.

Further provided are methods of treating an individual having a mitochondrial-associated disorder according to any one of the methods described above, wherein the treatment is based on presence (such as a level, for example a low level) of a biomarker in the individual including, but not limited to, 3-methylglutaconate, acylcarnitine, amino acids, ammonia, carnitine, citric acid cycle intermediates, coenzyme Q10, copper, creatine, creatinine, creatinine kinase, dicarboxylic acid, electrolytes, ethylmalonate, free fatty acids, very long chain fatty acids, glucose, ketones, lactate, myoglobin, neurotransmitters, organic acids, pyruvate, uric acid, red blood cells, and white blood cells. In some embodiments, the methods further comprise determining presence (such as a level, for example a low level) of a biomarker in the individual including, but not limited to, 3-methylglutaconate, acylcarnitine, amino acids, ammonia, carnitine, citric acid cycle intermediates, coenzyme Q10, copper, creatine, creatinine, creatinine kinase, dicarboxylic acid, electrolytes, ethylmalonate, free fatty acids, very long chain fatty acids, glucose, ketones, lactate, myoglobin, neurotransmitters, organic acids, pyruvate, uric acid, red blood cells, and white blood cells. In some embodiments, the methods further comprise selecting the individual for treatment based on presence (such as a level, for example a low level) of a biomarker in the individual including, but not limited to, 3-methylglutaconate, acylcarnitine, amino acids, ammonia, carnitine, citric acid cycle intermediates, coenzyme Q10, copper, creatine, creatinine, creatinine kinase, dicarboxylic acid, electrolytes, ethylmalonate, free fatty acids, very long chain fatty acids, glucose, ketones, lactate, myoglobin, neurotransmitters, organic acids, pyruvate, uric acid, red blood cells, and white blood cells. In some embodiments, the presence of a biomarker is assessed from a blood sample. In some embodiments, the presence of a biomarker is assessed from a urine sample.

Further provided are methods of treating an individual having a mitochondrial-associated disorder according to any one of the methods described above, wherein the treatment is based on mutation status of a biomarker in the individual including, but not limited to, MT-ATP6, MT-ND1, MT-ND2, MT-ND3, MT-ND5, MT-ND6, MT-TL1, MT-TH, MT-TV, NDUFS1, NDUFS2, NDUFS3, NDUFS4, NDUFS7, NDUFS8, or SURF1. In some embodiments, the methods further comprise determining mutation status of a biomarker in the individual including, but not limited to, MT-ATP6, MT-ND1, MT-ND2, MT-ND3, MT-ND5, MT-ND6, MT-TL, MT-TH, MT-TV, NDUFS1, NDUFS2, NDUFS3, NDUFS4, NDUFS7, NDUFS8, or SURF1. In some embodiments, the methods further comprise selecting the individual for treatment based on mutation status of a biomarker in the individual including, but not limited to, MT-ATP6, MT-ND1, MT-ND2, MT-ND3, MT-ND5, MT-ND6, MT-TL1, MT-TH, MT-TV, NDUFS1, NDUFS2, NDUFS3, NDUFS4, NDUFS7, NDUFS8, or SURF1.

These and other aspects and advantages of the present invention will become apparent from the subsequent detailed description and the appended claims. It is to be understood that one, some, or all of the properties of the various embodiments described herein may be combined to form other embodiments of the present invention.

### Definitions

As used herein “MELAS syndrome” refers to mitochondrial encephalomyopathy, lactic acidosis, and stroke-like episodes syndrome.

As used herein “NARP syndrome” refers to neuropathy, ataxia, and retinitis pigmentosa syndrome.

As used herein, “treatment” or “treating” is an approach for obtaining beneficial or desired results including clinical results. For purposes of this invention, beneficial or desired clinical results include, but are not limited to, alleviating one or more symptoms of a disease, such as a mitochondrial-associated disorder, reducing one or more symptoms of a disease, such as a mitochondrial-associated disorder, preventing one or more symptoms of a disease, such as a mitochondrial-associated disorder, treating one or more symptoms of a disease, such as a mitochondrial-associated disorder, ameliorating one or more symptoms of a disease, such as a mitochondrial-associated disorder, delaying onset of one or more symptoms associated with having a disease, such as a mitochondrial-associated disorder, diminishing the extent of one or more symptoms of a disease, such as a mitochondrial-associated disorder, stabilizing the disease, such as a mitochondrial-associated disorder (e.g., preventing or delaying the worsening of the disease), delaying or slowing the progression of the disease, such as a mitochondrial-associated disorder, ameliorating one or more symptoms of a disease, such as a mitochondrial-associated disorder, decreasing the dose of one or more other medications and/or treatments required to treat the disease, such as a mitochondrial-associated disorder, increasing the quality of life of the individual, and/or prolonging survival of the individual. Also encompassed by “treatment” is a reduction of a pathological consequence of a mitochondrial-associated disorder. The methods of the invention contemplate any one or more of these aspects of treatment.

The term “individual” refers to a mammal and includes, but is not limited to, human, bovine, horse, feline, canine, rodent, or primate. In some embodiments, the individual is human.

As used herein, an “at risk” individual is an individual who is at risk of developing a disease, such as a mitochondrial-associated disorder. An individual “at risk” may or may not have a detectable disease, such as a mitochondrial-associated disorder, and may or may not have displayed detectable symptoms or indications of a disease, such as a mitochondrial-associated disorder, prior to the treatment methods described herein. “At risk” denotes that an individual has one or more so-called risk factors, which are measurable parameters that correlate with development of a disease, such as a mitochondrial-associated disorder, which are described herein. An individual having one or more of these risk factors has a higher probability of developing a disease, such as a mitochondrial-associated disorder, than an individual without these risk factor(s).

As used herein, “delaying” the development of a disease, such as a mitochondrial-associated disorder means to defer, hinder, slow, retard, stabilize, and/or postpone development of the disease, such as a mitochondrial-associated disorder. This delay can be of varying lengths of time, depending on the history of the disease, such as a mitochondrial-associated disorder, and/or individual being treated. As is evident to one skilled in the art, a sufficient or significant delay can, in effect, encompass prevention, in that the individual does not develop or further develop the disease, such as a mitochondrial-associated disorder. A method that “delays” development of a disease, such as a mitochondrial-associated disorder, is a method that reduces probability of the disease development in a given time frame and/or reduces the extent of the disease in a given time frame, when compared to not using the method. Such comparisons are typically based on clinical studies, using a statistically significant number of subjects. Disease development, such as development of a mitochondrial-associated disorder, can be detectable using standard methods, including, but not limited to, audiogram, magnetic resonance imaging, computed tomography, magnetic resonance spectroscopy, electroencephalography, electrocardiography, echocardiography, electroretinography, immunohistochemistry, and assessment of a biomarker. Development may also refer to disease progression, such as progression of a mitochondrial-associated disorder, that may be initially undetectable and includes occurrence, recurrence, and onset.

The term “effective amount” used herein refers to an amount of a compound or composition sufficient to treat a specified disorder, condition, or disease, such as ameliorate, palliate, lessen, and/or delay one or more of its symptoms. For example, in reference to a mitochondrial-associated disorder, an effective amount comprises an amount sufficient to delay development of a mitochondrial-associated disorder. In some embodiments, the effective amount is an amount sufficient to prevent or delay recurrence. An effective amount can be administered in one or more administrations. For example, in the case of mitochondrial-associated disorders, the effective amount of the drug or composition may: (i) inhibit, retard, slow to some extent and preferably stop muscular dysfunction; (ii) inhibit, retard, slow to some extent and preferably stop neurological dysfunction; (iii) inhibit, retard, slow to some extent respiratory dysfunction; (iv) inhibit, retard, slow to some extent morbidity; (vi) prevent or delay occurrence and/or recurrence of a mitochondrial-associated disorder; and/or (vii) relieve to some extent one or more of the symptoms associated with having a mitochondrial-associated disorder.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the pharmaceutical composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein, the term “nab” stands for nanoparticle albumin-bound. For example, nab-sirolimus is a nanoparticle albumin-bound formulation of sirolimus. Nab-sirolimus is also known as nab-rapamycin, which has been previously described in, for example, WO2008109163, WO2014151853, WO2008137148, and WO2012149451.

As used herein, the term “mutation status” refers to the status of a gene sequence (e.g., containing a mutation) relative to a wildtype or reference gene sequence.

It is understood that aspects and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X.”

As used herein and in the appended claims, the singular forms “a,” “or,” and “the” include plural referents unless the context clearly dictates otherwise.

### Mitochondrial-Associated Disorders

As used herein, the term “mitochondrial-associated disorder” refers to any disease or disorder caused by dysfunction of a mitochondrion. Mitochondrial-associated disorders can cause a complex variety of symptoms. Symptoms of mitochondrial-associated disorders include, for example, muscle weakness, muscle cramps, seizures, food reflux, learning disabilities, deafness, short stature, paralysis of eye muscles, diabetes, cardiac problems, and stroke-like episodes. Symptoms of mitochondrial-associated disorders can range in severity from life-threatening to almost unnoticeable.

An individual having a mitochondrial-associated disorder can be classified in one or more subsets of mitochondrial-associated disorders based on genotype, phenotypic presentation, and/or one or more symptoms. In some embodiments, the individual having a mitochondrial-associated disorder has one or more of the following: an ataxia, a kidney disorder, a liver disorder, a metabolic disorder, a myopathy, a neuropathy, a myelopathy, an encephalopathy, an oxidative phosphorylation disorder, an aging disorder, an autism spectrum disorder, a chronic inflammatory disorder, or a fatty acid oxidation disorder. In some embodiments, the individual having a mitochondrial-associated disorder has one or more of the following: an ataxia, a kidney disorder, a liver disorder, a metabolic disorder, a myopathy, a neuropathy, a myelopathy, an encephalopathy, or an oxidative phosphorylation disorder. In some embodiments, the individual having a mitochondrial-associated disorder has one or more of the following: an aging disorder, an autism spectrum disorder, a chronic inflammatory disorder, diabetes mellitus, or a fatty acid oxidation disorder. In some embodiments, the individual having a mitochondrial-associated disorder has at least an ataxia. In some embodiments, the individual having a mitochondrial-associated disorder has at least a myelopathy and an encephalopathy. In some embodiments, the individual having a mitochondrial-associated disorder has at least a neuropathy, a myelopathy, and an encephalopathy. In some embodiments, the individual having a mitochondrial-associated disorder has at least a myopathy and a neuropathy.

In some embodiments, the individual having a mitochondrial-associated disorder has a mitochondrial DNA mutation-associated disorder. Individuals with mitochondrial DNA mutation-associated disorders include those having a mutation in a mitochondrial gene. In some embodiments, the individual having a mitochondrial-associated disorder has a nuclear DNA mutation-associated disorder. Individuals with nuclear DNA mutation-associated disorders include those individuals having a mutation in a nuclear gene. In some embodiments, the individual having a mitochondrial-associated disorder has an X chromosome mutation-associated disorder. Individuals with X chromosome mutation-associated disorders include those individuals having a mutation in the X chromosome.

Examples of mitochondrial-associated disorders include, but are not limited to: Leigh syndrome; MELAS syndrome; NARP syndrome; myoclonus epilepsy with ragged-red fibers (MERFF); chronic progressive external ophthalmoplegia (CPEO); Kearns Sayre syndrome (KSS); mitochondrial neurogastrointestinal encephalopathy (MNGIE); Friedreich's ataxia; amyotrophic lateral sclerosis (ALS); Huntington's disease; Parkinson's Disease; macular degeneration; epilepsy; Alzheimer's; Leber's hereditary optic neuropathy (LHON); progressive external ophthalmoplegia (PEO); diabetes mellitus; diabetes mellitus and deafness (DAD); Pearson syndrome; Alpers disease (progressive infantile poliodystrophy); ataxia neuropathy spectrum; autism spectrum disorder; Barth syndrome (lethal infantile cardiomyopathy); carnitine-acyl-carnitine translocase deficiency; carnitine deficiency; carnitine palmitoyltransferase I deficiency; carnitine palmitoyltransferase II: cerebral creatine deficiency syndromes; coenzyme Q10 deficiency; complex I deficiency; complex II deficiency; complex III deficiency; complex IV/COX deficiency; complex V deficiency; infantile myopathy and lactic acidosis; leukoencephalopathy with brain stem and spinal cord involvement and lactate elevation (LBSL); long-chain 3-hydroxyacyl-CoA dehydrogenase deficiency (LCHAD); long-chain acyl-CoA dehydrogenase deficiency (LCAD); Luft disease; medium-chain acyl-CoA dehydrogenase deficiency (MCAD); mtDNA depletion syndrome (MDS); multiple acyl-CoA dehydrogenase deficiency (MADD); myoclonic epilepsy myopathy sensory ataxia (MEMSA); pyruvate carboxylase deficiency; pyruvate dehydrogenase complex deficiency (PDCD); short-chain acyl-CoA dehydrogenase deficiency (SCAD); and very long-chain acyl-CoA dehydrogenase deficiency (VLCAD).

### Leigh Syndrome

In some embodiments, the individual having a mitochondrial-associated disorder has Leigh syndrome. Leigh syndrome, also referred to as subacute necrotizing encephalomyelopathy, is a mitochondrial-associated neurodegenerative disorder, primarily affecting infants and young children. Generally. Leigh syndrome is characterized by lesions in the basal ganglia, thalamus, and brainstem. Symptoms of Leigh syndrome include, but are not limited to, psychomotor retardation, seizures, nystagmus, ophthalmoparesis, optic atrophy, ataxia, dystonia, respiratory failure, polyneuropathy or myoneuropathy, diabetes, short stature, hypertrichosis, cardiomyopathy, anemia, renal failure, vomiting, and diarrhea.

Leigh syndrome can be caused by mutations in one of more than 75 different genes. Most genes are found in nuclear DNA, while some are found in the mitochondria within cells (mitochondrial DNA, mtDNA). Most individuals with Leigh syndrome have a mutation in nuclear DNA, with about 20% having a mutation in mtDNA. Disruption of NADH:ubiquinone (found in mitochondrial protein complex 1) is the most common cause of Leigh syndrome, accounting for approximately 30% of cases. At least 25 genes found in the formation of mitochondrial complex 1 proteins are found in either nuclear DNA or mtDNA and have been associated with Leigh syndrome. Disruption of mitochondrial protein complex IV, also called cytochrome c oxidase or COX, is also a common cause of Leigh's syndrome, underlying approximately 15% percent of cases. One of the most frequently mutated genes SURF 1, found in nuclear DNA, provides instructions for making a protein that helps to assemble the COX protein complex (complex IV), which provides energy to be used in the process of generating ATP. Mutations in the SURF1 gene can result if the absence of functional SURF protein, which then reduces the formation of normal COX complex, ultimately impairing mitochondrial energy production. The most common mtDNA mutation in Leigh's syndrome affects the MT-ATP 6 gene, which provides instructions for making a piece of complex V, also known as the ATP synthase protein complex that generates ATP. This mutation is found in approximately 10% of patients with Leigh's syndrome. Other mtDNA mutations associated with this syndrome decrease the activity of other oxidative phosphorylation protein complexes or lead to reduced formation of mitochondrial proteins, all of which impair mitochondrial energy production.

Other gene mutations associated with this disease disrupt the activity of one or more oxidative phosphorylation protein complexes or affect additional steps related to energy production. Mutations in genes that direct the replication of mtDNA or the production of mitochondrial proteins can also disrupt mitochondrial energy production.

Genetic mutations that are associated with the development of Leigh syndrome include, but are not limited to, SURF1, MT-ATP6, MT-ND2, MT-ND3, MT-ND5, MT-ND6, and LRPPRC. In some embodiments, the genetic mutation is a mutation of a mitochondrial gene. In some embodiments, the genetic mutation is a mutation of a nuclear gene. In some embodiments, the genetic mutation is a thymine to guanine mutation at nucleic acid 8993 of MT-ATP6.

In some embodiments, the individual having Leigh syndrome has infantile onset Leigh syndrome. In some embodiments, the individual having infantile onset Leigh syndrome exhibits onset symptoms between the ages of three months and two years. In some embodiments, the individual having Leigh syndrome has juvenile onset Leigh syndrome. In some embodiments, the individual having juvenile onset Leigh syndrome exhibits onset symptoms after two years of age. In some embodiments, the individual having infantile onset Leigh syndrome exhibits onset symptoms between the ages of three months and two years. In some embodiments, the individual having Leigh syndrome has adult onset Leigh syndrome. In some embodiments, the individual having adult onset Leigh syndrome exhibits onset symptoms after 10 years of age.

In some embodiments, the individual having Leigh syndrome exhibits symptoms as an infant or a child, such as at less than or two years of age. In some embodiments, the allosteric mTOR inhibitor can be administered either at the time of onset or more than 1, 2, 3, 4, 5, 6, 7, 8, 9, 10, 11, 12, 13, 14, 15, 16, 17, 18, 19, 20, 21, 22, 23, or 24 months after the time of onset.

### MELAS Syndrome

In some embodiments, the individual having a mitochondrial-associated disorder has MELAS syndrome. MELAS syndrome is a mitochondrial-associated disorder that affects the nervous system and muscles. Symptoms of MELAS syndrome typically appear in childhood, following a period of normal development. Early symptoms of MELAS syndrome include, but are not limited to, muscle weakness, muscle pain, recurrent headaches, loss of appetite, vomiting, and seizures. Symptoms of MELAS syndrome include, but are not limited to, normal early development, seizures, stroke-like episodes, growth retardation/short stature, hearing loss, limb weakness, aphasia, cortical blindness, ataxia, tremor, exercise intolerance, migraines, vomiting, diabetes, cerebral lesions, and unexplained neurologic/psychiatric disorders. In some embodiments, the individual having MELAS syndrome experiences stroke-like episodes. These stroke-like episodes may involve temporary muscle weakness on one side of the body (hemiparesis), altered consciousness, vision abnormalities, seizures, and severe headaches resembling migraines. Repeated stroke-like episodes can progressively damage the brain leading to vision loss, problems with movement, and a loss of intellectual function (dementia).

In some embodiments, the individual having MELAS syndrome exhibits a buildup of lactic acid, a condition called lactic acidosis. Increased acidity in the blood can lead to vomiting, abdominal pain, extreme tiredness (fatigue), muscle weakness, and difficulty breathing. The individual having MELAS syndrome may also experience involuntary muscle spasms (myoclonus), impaired muscle coordination (ataxia), hearing loss, heart and kidney issues, diabetes, and hormonal imbalances.

Genetic mutations that are associated with the development of MELAS syndrome include, but are not limited to, MT-ND1, MT-ND5, MT-TH, MT-TL1, and MT-TV. In some embodiments, the genetic mutation is a mutation of a mitochondrial gene.

Generally, MELAS syndrome is maternally inherited. However, somatic mutations can arise in an individual that cause MELAS syndrome. Thus, both are contemplated in the present application.

Diagnosing MELAS syndrome may include analysis of lactic acid and pyruvate levels in an individual. In some embodiments, the individual having MELAS syndrome exhibits a high level of arterial lactate and pyruvate. In some embodiments, the individual having MELAS syndrome exhibits a high level of cerebral spinal fluid (CSF) lactate. In some embodiments, the individual having MELAS syndrome exhibits an increased ratio of lactate to pyruvate.

Diagnosing MELAS syndrome may include analysis of muscle tissue. In some embodiments, the individual having MELAS syndrome exhibits ragged red fibers.

Diagnosing MELAS syndrome may include histological analysis of muscle tissue. In some embodiments, the individual having MELAS syndrome exhibits ragged red fibers. In some embodiments, the individual having MELAS syndrome exhibits an increased number of structurally abnormal mitochondria. In some embodiments, the individual having MELAS syndrome exhibits an increased number of structurally abnormal mitochondria in endothelial and smooth muscle cells of arterioles and small arteries.

In some embodiments, the individual having MELAS syndrome exhibits overall progressive neurological deficits, punctuated with periods of relapse and remission. In some embodiments, the individual having MELAS syndrome has a life span of less than 40 years of age.

### NARP Syndrome

In some embodiments, the individual having a mitochondrial-associated disorder has NARP syndrome. NARP syndrome is mitochondrial-associated disorder that affects the nervous system. In some embodiments, the individual having NARP syndrome exhibits symptoms in childhood or early adulthood. Symptoms of NARP syndrome include, but are not limited to, sensory neuropathy (numbness, tingling, and pain in the arms and legs), muscle weakness, exercise intolerance, ataxia, vision loss, learning disabilities, development delays, seizures, hearing loss, and cardiac conduction defects. In some embodiments, the individual having NARP syndrome has dementia. In some embodiments, the individual having NARP syndrome does not exhibit symptoms of NARP syndrome. In some embodiments, the individual having NARP syndrome exhibits a mild degree of symptoms associated with NARP syndrome. In some embodiments, the individual with NARP syndrome exhibits a severe degree of symptoms associated with NARP syndrome.

Genetic mutations that are associated with the development of NARP syndrome include, but are not limited to, MT-ATP6. In some embodiments, the individual having NARP syndrome exhibits a MT-ATP6 mutation in less than 90% of mitochondria. In some embodiments, the individual having NARP syndrome exhibits a MT-ATP6 mutation in 70-90% of mitochondria.

Diagnosing NARP syndrome may include a neurological exam, such as electromyography, nerve conduction testing, a magnetic resonance imaging (MRI) test, or a magnetic resonance (MR) spectroscopy test.

Diagnosing NARP syndrome may include histological analysis of muscle tissue. In some embodiments, the individual having NARP syndrome exhibits ragged red fibers.

Diagnosing NARP syndrome may include genetic testing.

### Methods of Treating Mitochondrial-Associated Disorders

The present invention provides methods of treating an individual (e.g., human) having a mitochondrial-associated disorder comprising administering to the individual an effective amount of an allosteric mTOR inhibitor (such as a limus drug). In some embodiments, the invention provides methods of treating an individual (e.g., human) having a mitochondrial-associated disorder comprising administering to the individual an effective amount of a composition comprising nanoparticles comprising an allosteric mTOR inhibitor and an albumin. In some embodiments, the invention provides methods of treating an individual (e.g, human) having a mitochondrial-associated disorder comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the weight ratio of albumin and limus drug in the nanoparticle composition is about 8:1. In some embodiments, the weight ratio of albumin and limus drug in the nanoparticle composition is about 8.5:1. In some embodiments, the weight ratio of albumin and limus drug in the nanoparticle composition is about 9:1. In some embodiments, the weight ratio of albumin and limus drug in the nanoparticle composition is about 10:1. In some embodiments, the weight ratio of albumin and limus drug in the nanoparticle composition is about 11:1. In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus.

In some embodiments, the individual having a mitochondrial-associated disorder has an ataxia. In some embodiments, the individual having a mitochondrial-associated disorder has a kidney disorder. In some embodiments, the individual having a mitochondrial-associated disorder has a liver disorder. In some embodiments, the individual having a mitochondrial-associated disorder has a metabolic disorder. In some embodiments, the individual having a mitochondrial-associated disorder has a myopathy. In some embodiments, the individual having a mitochondrial-associated disorder has a neuropathy. In some embodiments, the individual having a mitochondrial-associated disorder has a myelopathy. In some embodiments, the individual having a mitochondrial-associated disorder has an encephalopathy. In some embodiments, the individual having a mitochondrial-associated disorder has an oxidative phosphorylation disorder. In some embodiments, the individual having a mitochondrial-associated disorder has an aging disorder. In some embodiments, the individual having a mitochondrial-associated disorder has an autism spectrum disorder. In some embodiments, the individual having a mitochondrial-associated disorder has a chronic inflammatory disorder. In some embodiments, the individual having a mitochondrial-associated disorder has diabetes mellitus. In some embodiments, the individual having a mitochondrial-associated disorder has a fatty acid oxidation disorder. In some embodiments, the individual having a mitochondrial-associated disorder has a mitochondrial DNA mutation-associated disorder. In some embodiments, the individual having a mitochondrial-associated disorder has an X chromosome mutation-associated disorder. In some embodiments, the individual having a mitochondrial-associated disorder has a nuclear DNA mutation-associated disorder. In some embodiments, the individual having a mitochondrial-associated disorder has Leigh syndrome. In some embodiments, the individual having a mitochondrial-associated disorder has maternally inherited Leigh syndrome. In some embodiments, Leigh syndrome is infantile onset Leigh syndrome. In some embodiments, the individual having a mitochondrial-associated disorder has juvenile onset Leigh syndrome. In some embodiments, the individual having a mitochondrial-associated disorder has adult onset Leigh syndrome. In some embodiments, the individual having a mitochondrial-associated disorder has MELAS syndrome. In some embodiments, the individual having a mitochondrial-associated disorder has NARP syndrome.

In some embodiments, the individual having a mitochondrial-associated disorder has one or more of the following: an ataxia, a kidney disorder, a liver disorder, a metabolic disorder, a myopathy, a neuropathy, a myelopathy, an encephalopathy, an oxidative phosphorylation disorder, an aging disorder, an autism spectrum disorder, a chronic inflammatory disorder, diabetes mellitus, a fatty acid oxidation disorder, a mitochondrial DNA mutation-associated disorder, an X chromosome mutation-associated disorder, a nuclear DNA mutation-associated disorder. In some embodiments, the individual having a mitochondrial-associated disorder has at least an ataxia. In some embodiments, the individual having a mitochondrial-associated disorder has at least a myelopathy and an encephalopathy. In some embodiments, the individual having a mitochondrial-associated disorder has at least a neuropathy, a myelopathy, and an encephalopathy. In some embodiments, the individual having a mitochondrial-associated disorder has at least a myopathy and a neuropathy.

In some embodiments, the individual having a mitochondrial-associated disorder has an immunohistochemically identified marker, such as presence of ragged red fibers or structurally abnormal mitochondria. In some embodiments, the individual having a mitochondrial-associated disorder has a high level of a lactate, such as lactic acid, and pyruvate, such as a high level of arterial lactate and pyruvate. In some embodiments, the individual having a mitochondrial-associated disorder has an increased ratio of lactate to pyruvate, such as a lactate to pyruvate ratio of at least 10:1. In some embodiments, the individual having a mitochondrial-associated disorder has mutation status in one or more of the following genes: LRPPRC, MT-ATP6, MT-ND1, MT-ND2, MT-ND3, MT-ND5, MT-ND6, MT-TL1, MT-TH, MT-TV, or SURF1.

In some embodiments, the individual having a mitochondrial-associated disorder has: an early stage mitochondrial-associated disorder, an advanced mitochondrial-associated disorder, a recurrent mitochondrial-associated disorder, or a mitochondrial-associated disorder in remission. In some embodiments, the individual having a mitochondrial-associated disorder is refractory to a prior therapy. In some embodiments, the individual having a mitochondrial-associated disorder is resistant to the treatment with a non-nanoparticle formulation of a therapeutic agent (such as non-nanoparticle formulation of an allosteric mTOR inhibitor, such as a limus drug).

The methods provided herein can be used to treat an individual (e.g., human) who has been diagnosed with or is suspected of having a mitochondrial-associated disorder. In some embodiments, the individual is human. In some embodiments, the individual is any of about 1, 2, 3, 4, 5, 6, 7, 8, 9, 10, 11, 12, 13, 14, 15, 16, 17, 18, 19, 20, 21, 22, 23, or 24 months old. In some embodiments, the individual is less than about 50, 45, 40, 35, 30, 25, 20, 15, 10, 9, 8, 7, 6, 5, 4, 3, 2, or 1 years of age. In some embodiments, the individual is any of about 1, 2, 3, 4, 5, 6, 7, 8, 9, 10, 11, 12, 13, 14, 15, 16, 17, 18, 19, 20, 21, 22, 23, or 24 months old at the age of onset of one or more mitochondrial-associated disorder symptoms. In some embodiments, the age of onset of one or more mitochondrial-associated disorder symptoms in the individual is any of about 1, 2, 3, 4, 5, 6, 7, 8, 9, 10, 11, 12, 13, 14, 15, 16, 17, 18, 19, 20, 21, 22, 23, or 24 months old. In some embodiments, the individual is a male. In some embodiments, the individual is a female.

In some embodiments, there is provided a method of treating an individual (such as human) having a mitochondrial-associated disorder having an ataxia comprising administering to the individual an effective amount of an allosteric mTOR inhibitor (such as a limus drug). In some embodiments, the invention provides methods of treating an individual (such as human) having a mitochondrial-associated disorder having an ataxia comprising administering to the individual an effective amount of a composition comprising nanoparticles comprising an allosteric mTOR inhibitor and an albumin. In some embodiments, the invention provides methods of treating an individual (such as human) having a mitochondrial-associated disorder having an ataxia comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor (such as a nanoparticle limus drug) is administered at a dosage of about 1 mg/m² to about 40 mg/m² (such as about any of 10 mg/m², 20 mg/m², or 30 mg/m²) weekly. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating an individual (such as human) having a mitochondrial-associated disorder having a myelopathy and an encephalopathy comprising administering to the individual an effective amount of an allosteric mTOR inhibitor (such as a limus drug). In some embodiments, the invention provides methods of treating an individual (such as human) having a mitochondrial-associated disorder having a myelopathy and an encephalopathy comprising administering to the individual an effective amount of a composition comprising nanoparticles comprising an allosteric mTOR inhibitor and an albumin. In some embodiments, the invention provides methods of treating an individual (such as human) having a mitochondrial-associated disorder having a myelopathy and an encephalopathy comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor (such as a nanoparticle limus drug) is administered at a dosage of about 1 mg/m² to about 40 mg/m² (such as about any of 10 mg/m², 20 mg/m², or 30 mg/m²) weekly. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating an individual (such as human) having a mitochondrial-associated disorder having a neuropathy, a myelopathy, and an encephalopathy comprising administering to the individual an effective amount of an allosteric mTOR inhibitor (such as a limus drug). In some embodiments, the invention provides methods of treating an individual (such as human) having a mitochondrial-associated disorder having a neuropathy, a myelopathy, and an encephalopathy comprising administering to the individual an effective amount of a composition comprising nanoparticles comprising an allosteric mTOR inhibitor and an albumin. In some embodiments, the invention provides methods of treating an individual (such as human) having a mitochondrial-associated disorder having a neuropathy, a myelopathy, and an encephalopathy comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor (such as a nanoparticle limus drug) is administered at a dosage of about 1 mg/m² to about 40 mg/m² (such as about any of 10 mg/m², 20 mg/m², or 30 mg/m²) weekly. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating an individual (such as human) having a mitochondrial-associated disorder having a myopathy and a neuropathy comprising administering to the individual an effective amount of an allosteric mTOR inhibitor (such as a limus drug). In some embodiments, the invention provides methods of treating an individual (such as human) having a mitochondrial-associated disorder having a myopathy and a neuropathy comprising administering to the individual an effective amount of a composition comprising nanoparticles comprising an allosteric mTOR inhibitor and an albumin. In some embodiments, the invention provides methods of treating an individual (such as human) having a mitochondrial-associated disorder having a myopathy and a neuropathy comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle 3) composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor (such as a nanoparticle limus drug) is administered at a dosage of about 1 mg/m² to about 40 mg/m² (such as about any of 10 mg/m², 20 mg/m², or 30 mg/m²) weekly. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating an individual (such as human) with Leigh syndrome comprising administering to the individual an effective amount of an allosteric mTOR inhibitor (such as a limus drug). In some embodiments, the invention provides methods of treating an individual (such as human) with Leigh syndrome comprising administering to the individual an effective amount of a composition comprising nanoparticles comprising an allosteric mTOR inhibitor and an albumin. In some embodiments, the invention provides methods of treating an individual (such as human) with Leigh syndrome comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor (such as a nanoparticle limus drug) is administered at a dosage of about 1 mg/m² to about 40 mg/m² (such as about any of 10 mg/m², 20 mg/m², or 30 mg/m²) weekly. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating an individual (such as human) with maternally inherited Leigh syndrome comprising administering to the individual an effective amount of an allosteric mTOR inhibitor (such as a limus drug). In some embodiments, the invention provides methods of treating an individual (such as human) with maternally inherited Leigh syndrome comprising administering to the individual an effective amount of a composition comprising nanoparticles comprising an allosteric mTOR inhibitor and an albumin. In some embodiments, the invention provides methods of treating an individual (such as human) with maternally inherited Leigh syndrome comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor (such as a nanoparticle limus drug) is administered at a dosage of about 1 mg/m² to about 40 mg/m² (such as about any of 10 mg/m², 20 mg/m², or 30 mg/m²) weekly. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating an individual (such as human) with infantile onset Leigh syndrome comprising administering to the individual an effective amount of an allosteric mTOR inhibitor (such as a limus drug). In some embodiments, the invention provides methods of treating an individual (such as human) with infantile onset Leigh syndrome comprising administering to the individual an effective amount of a composition comprising nanoparticles comprising an allosteric mTOR inhibitor and an albumin. In some embodiments, the invention provides methods of treating an individual (such as human) with infantile onset Leigh syndrome comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor (such as a nanoparticle limus drug) is administered at a dosage of about 1 mg/m² to about 40 mg/m² (such as about any of 10 mg/m², 20 mg/m², or 30 mg/m²) weekly. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating an individual (such as human) with juvenile onset Leigh syndrome comprising administering to the individual an effective amount of an allosteric mTOR inhibitor (such as a limus drug). In some embodiments, the invention provides methods of treating an individual (such as human) with juvenile onset Leigh syndrome comprising administering to the individual an effective amount of a composition comprising nanoparticles comprising an allosteric mTOR inhibitor and an albumin. In some embodiments, the invention provides methods of treating an individual (such as human) with juvenile onset Leigh syndrome comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 1 mg/m² to about 100 mg/m², including, for example, about 5 mg/m² to about 100 mg/m² (such as about any of 10 mg/m², 20 mg/m², or 30 mg/m²). In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor (such as a nanoparticle limus drug) is administered at a dosage of about 1 mg/m² to about 40 mg/m² (such as about any of 10 mg/m², 20 mg/m², or 30 mg/m²) weekly. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating an individual (such as human) with adult onset Leigh syndrome comprising administering to the individual an effective amount of an allosteric mTOR inhibitor (such as a limus drug). In some embodiments, the invention provides methods of treating an individual (such as human) with adult onset Leigh syndrome comprising administering to the individual an effective amount of a composition comprising nanoparticles comprising an allosteric mTOR inhibitor and an albumin. In some embodiments, the invention provides methods of treating an individual (such as human) with adult onset Leigh syndrome comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor (such as a nanoparticle limus drug) is administered at a dosage of about 1 mg/m² to about 40 mg/m² (such as about any of 10 mg/m², 20 mg/m², or 30 mg/m²) weekly. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating an individual (such as human) with MELAS syndrome comprising administering to the individual an effective amount of an allosteric mTOR inhibitor (such as a limus drug). In some embodiments, the invention provides methods of treating an individual (such as human) with MELAS syndrome comprising administering to the individual an effective amount of a composition comprising nanoparticles comprising an allosteric mTOR inhibitor and an albumin. In some embodiments, the invention provides methods of treating an individual (such as human) with MELAS syndrome comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor (such as a nanoparticle limus drug) is administered at a dosage of about 1 mg/m² to about 40 mg/m² (such as about any of 10 mg/m², 20 mg/m², or 30 mg/m²) weekly. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating an individual (such as human) with NARP syndrome comprising administering to the individual an effective amount of an allosteric mTOR inhibitor (such as a limus drug). In some embodiments, the invention provides methods of treating an individual (such as human) with NARP syndrome comprising administering to the individual an effective amount of a composition comprising nanoparticles comprising an allosteric mTOR inhibitor and an albumin. In some embodiments, the invention provides methods of treating an individual (such as human) with NARP syndrome comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor (such as a nanoparticle limus drug) is administered at a dosage of about 1 mg/m² to about 40 mg/m² (such as about any of 10 mg/m², 20 mg/m², or 30 mg/m²) weekly. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

The methods provided herein may be practiced in an adjuvant setting. In some embodiments, the method is practiced in a neoadjuvant setting. i.e., the method may be carried out before the primary/definitive therapy. In some embodiments, the method is used to treat an individual who has previously been treated. In some embodiments, the individual has not previously been treated. In some embodiments, the method is used as a first line therapy. In some embodiments, the method is used as a second line therapy. In some embodiments, the individual has not been previously treated with an allosteric mTOR inhibitor. In some embodiments, the individual has not been previously treated with a limus drug. In some embodiments, the individual has not been previously treated with sirolimus. In some embodiments, the individual has not been previously treated with a composition comprising nanoparticles comprising an allosteric mTOR inhibitor and an albumin. In some embodiments, the individual has not been previously treated with a composition comprising nanoparticles comprising a limus drug and an albumin. In some embodiments, the individual has not been previously treated with a composition comprising nanoparticles comprising sirolimus and an albumin. In some embodiments, the individual has not been previously treated with nab-sirolimus.

In some embodiments, there is provided a method of prolonging time to progression of a mitochondrial-associated disorder in an individual, comprising administering to the individual an effective amount of an allosteric mTOR inhibitor. In some embodiments, there is provided a method of prolonging time to progression of a mitochondrial-associated disorder in an individual, comprising administering to the individual an effective amount of a composition comprising nanoparticles comprising an allosteric mTOR inhibitor and an albumin. In some embodiments, the method prolongs the time to progression of a mitochondrial-associated disorder by at least any of about 1, 2, 3, 4, 5, 6, 7, 8, 9, 10, 11, or 12 weeks. In some embodiments, the allosteric mTOR inhibitor is a limus drug. In some embodiments, the limus drug is sirolimus. In some embodiments, the allosteric mTOR inhibitor comprises nab-sirolimus. In some embodiments, the allosteric mTOR inhibitor is nab-sirolimus.

In some embodiments, there is provided a method of prolonging survival of an individual having a mitochondrial-associated disorder, comprising administering to the individual an effective amount of an allosteric mTOR inhibitor. In some embodiments, there is provided a method of prolonging survival of an individual having a mitochondrial-associated disorder, comprising administering to the individual an effective amount of a composition comprising nanoparticles comprising an allosteric mTOR inhibitor and an albumin. In some embodiments, the method prolongs the survival of the individual by at least any of 1, 2, 3, 4, 5, 6, 7, 8, 9, 10, 11, 12, 18, or 24 month. In some embodiments, the allosteric mTOR inhibitor is a limus drug. In some embodiments, the limus drug is sirolimus. In some embodiments, the allosteric mTOR inhibitor comprises nab-sirolimus. In some embodiments, the allosteric mTOR inhibitor is nab-sirolimus.

In some embodiments, there is provided a method of alleviating one or more symptoms in an individual having a mitochondrial-associated disorder, comprising administering to the individual an effective amount of an allosteric mTOR inhibitor. In some embodiments, there is provided a method of alleviating one or more symptoms in an individual having a mitochondrial-associated disorder, comprising administering to the individual an effective amount of a composition comprising nanoparticles comprising an allosteric mTOR inhibitor and an albumin. In some embodiments, the allosteric mTOR inhibitor is a limus drug. In some embodiments, the limus drug is sirolimus. In some embodiments, the allosteric mTOR inhibitor comprises nab-sirolimus. In some embodiments, the allosteric mTOR inhibitor is nab-sirolimus.

In some embodiments, there is provided a method of improving the quality of life in an individual having a mitochondrial-associated disorder, comprising administering to the individual an effective amount of an allosteric mTOR inhibitor. In some embodiments, there is provided a method of improving the quality of life in an individual having a mitochondrial-associated disorder, comprising administering to the individual an effective amount of a composition comprising nanoparticles comprising an allosteric mTOR inhibitor and an albumin. In some embodiments, the allosteric mTOR inhibitor is a limus drug. In some embodiments, the limus drug is sirolimus. In some embodiments, the allosteric mTOR inhibitor comprises nab-sirolimus. In some embodiments, the allosteric mTOR inhibitor is nab-sirolimus.

In some embodiments, the individual has been previously treated for a mitochondrial-associated disorder (also referred to as the “prior therapy”). In some embodiments, the individual is resistant to treatment of a mitochondrial-associated disorder with other agents (such as non-nanoparticle formulations of allosteric mTOR inhibitors). In some embodiments, the individual is initially responsive to treatment of a mitochondrial-associated disorder with other agents but has progressed after treatment.

In some embodiments, the individual is resistant to the prior therapy.

In some embodiments, the individual is unsuitable to continue with the prior therapy, for example, due to failure to respond and/or due to toxicity.

In some embodiments, the individual is non-responsive to the prior therapy.

In some embodiments, the individual is partially responsive to the prior therapy or exhibits a less desirable degree of responsiveness.

Also provided are pharmaceutical compositions comprising an allosteric mTOR inhibitor (such as limus drug, for example sirolimus) for use in any of the methods of treating an individual having a mitochondrial-associated disorder described herein.

Also provided are pharmaceutical compositions comprising nanoparticles comprising an allosteric mTOR inhibitor (such as limus drug, for example sirolimus) for use in any of the methods of treating an individual having a mitochondrial-associated disorder described herein. In some embodiments, the nanoparticle compositions comprise nanoparticles comprising an allosteric mTOR inhibitor (such as limus drug, for example sirolimus) and albumin (such as human albumin).

### Use of Biomarkers for Treating Mitochondrial-Associated Disorders

The present invention in one aspect provides methods of treating an individual having a mitochondrial-associated disorder based on the individual having one or more biomarkers, such as mutation status of a gene, activity level of an enzyme or coenzyme, or presence (such as a level) of a biomarker.

In some embodiments, there is provided a method of treating an individual having a mitochondrial-associated disorder comprising administering to the individual an effective amount of an allosteric mTOR inhibitor (such as a limus drug), wherein the individual is selected for treatment based on the individual having a mutation status in a gene. In some embodiments, there is provided a method of treating an individual having a mitochondrial-associated disorder comprising administering to the individual an effective amount of a composition comprising nanoparticles comprising an allosteric mTOR inhibitor (such as a limus drug) and an albumin, wherein the individual is selected for treatment based on the individual having a mutation status in a gene. In some embodiments, the gene is selected from LRPPRC, MT-ATP6, MT-ND1, MT-ND2, MT-ND3, MT-ND5, MT-ND6, MT-TL1, MT-TH, MT-TV, NDUFS1, NDUFS2, NDUFS3, NDUFS4, NDUFS7, NDUFS8, or SURF1. In some embodiments, the mutation status is identified in a sample from the individual via exome sequencing. In some embodiments, the mutation status is identified in a sample from the individual via sequencing of mitochondrial genes. In some embodiments, the mutation status is identified in a sample from the individual via sequencing of nuclear genes. In some embodiments, the mutation status of a gene is assessed using next-generation sequencing. In some embodiments, the mutation status of a gene isolated from a blood sample is assessed using next-generation sequencing. In some embodiments, the mutation status is identified in a sample from the individual via tissue biopsy mutation analysis. In some embodiments, the mutation status is identified in a sample from the individual via fluorescence in-situ hybridization. In some embodiments, the sample is a blood sample. In some embodiments, the sample is a cerebral spinal fluid sample. In some embodiments, the sample is obtained prior to initiation of the treatment methods described herein. In some embodiments, the sample is obtained after initiation of the treatment methods described herein.

In some embodiments, the gene is selected from LRPPRC, MT-ATP6, MT-ND1, MT-ND2, MT-ND3, MT-ND5, MT-ND6, MT-TL1, MT-TH, MT-TV, SURF1, ACAD9, ACADM, ACADVL, ANT1, APOPT1, APTX, ATP5A1, ATP5E, ATPAF2, B17.2L, BCS1L, C10ORF2, C12ORF62, C20ORF7, CABC1, COA3, COA5, COA6, COQ2, COQ4, COQ6, COQ7, COQ8, COQ9, COZ6B1, COX8A, COX 10, COX14, COX15, COX20, CPT1A, CPT2, DARS2, DGUOK, DLAT, ETFA, ETFB, ETFDH, FASTKD2, FOXRED1, FXN, GAMT, GATM, GFM1, HADH, HADHA, HRPAP20, LCAD, MPV17, MT-ATP8, MT-CO1, MT-CO2, MT-CO3, MT-CYB, MT-FMT, MT-ND4, MT-TE, MT-TK, MT-TS1, MT-TS2, NDUFA1, NDUFA2, NDUFA9, NDUFA10, NDUFA11, NDUFA12, NDUFAF1, NDUFAF2, NDUFAF3, NDUFAF4, NDUFAF5, NDUFAF6, NDUFB3, NDUFB9, NDUFS1, NDUFS2, NDUFS3, NDUFS4, NDUFS6, NDUFS7, NDUFS8, NDUFV1, NDUFV2, NUBPL, PARK2, PC, PDH, PDHA1, PDHB, PDHX, PDP1, PDSS1, PDSS2, PEO1, PET100, POLG, RRM2B, SCO1, SCO2, SDHA, SDHAF1, SDHD, SLC6A8, SLC25A4, SLC25A20, SLC22A5, SUCLA2, TACO1, TK2, TMEM70, TP, TAZ, TWINKLE, or TYMP.

In some embodiments, the mutation status in a gene is present in less than about 95%, 90%, 85%, 80%, 75%, 70%, 65%, 60%, 55%, 50%, 45%, 40%, 35%, 30%, 25%, 20%, 15%, 10%, or 5% of all mitochondria assessed. In some embodiments, the mutation status in a gene is present in about 70% to about 90% of all mitochondria assessed.

In some embodiments, there is provided a method of treating Leigh syndrome in an individual comprising administering to the individual an effective amount of an allosteric mTOR inhibitor (such as a limus drug), wherein the individual is selected for treatment based on the individual having a mutation status in SURF1. In some embodiments, there is provided a method of treating Leigh syndrome in an individual comprising administering to the individual an effective amount of an allosteric mTOR inhibitor (such as a limus drug), wherein the individual is selected for treatment based on the individual having a mutation status in MT-ATP6. In some embodiments, there is provided a method of treating Leigh syndrome in an individual comprising administering to the individual an effective amount of a composition comprising nanoparticles comprising an allosteric mTOR inhibitor (such as a limus drug) and an albumin, wherein the individual is selected for treatment based on the individual having a mutation status in SURF1. In some embodiments, there is provided a method of treating Leigh syndrome in an individual comprising administering to the individual an effective amount of a composition comprising nanoparticles comprising an allosteric mTOR inhibitor (such as a limus drug) and an albumin, wherein the individual is selected for treatment based on the individual having a mutation status in MT-ATP6. In some embodiments, the mutation status in MT-ATP6 is a thymine to guanine mutation in nucleic acid position 8993.

In some embodiments, there is provided a method of treating MELAS syndrome in an individual comprising administering to the individual an effective amount of an allosteric mTOR inhibitor (such as a limus drug), wherein the individual is selected for treatment based on the individual having a mutation status in MT-TL1. In some embodiments, there is provided a method of treating MELAS syndrome in an individual comprising administering to the individual an effective amount of a composition comprising nanoparticles comprising an allosteric mTOR inhibitor (such as a limus drug) and an albumin, wherein the individual is selected for treatment based on the individual having a mutation status in MT-TL1.

In some embodiments, there is provided a method of treating NARP syndrome in an individual comprising administering to the individual an effective amount of an allosteric mTOR inhibitor (such as a limus drug), wherein the individual is selected for treatment based on the individual having a mutation status in MT-ATP6. In some embodiments, there is provided a method of treating NARP syndrome in an individual comprising administering to the individual an effective amount of a composition comprising nanoparticles comprising an allosteric mTOR inhibitor (such as a limus drug) and an albumin, wherein the individual is selected for treatment based on the individual having a mutation status in MT-ATP6. In some embodiments, the individual is selected based on having a mutation status in MT-ATP6 in less than about 90% of mitochondria. In some embodiments, the individual is selected based on having a mutation status in MT-ATP6 in about 70% to about 90% of mitochondria. In some embodiments, the individual is selected based on having a mutation status in MT-ATP6 in less than about 70% of mitochondria.

In some embodiments, there is provided a method of treating an individual having a mitochondrial-associated disorder comprising administering to the individual an effective amount of an allosteric mTOR inhibitor (such as a limus drug), wherein the individual is selected for treatment based on the individual having a tissue biomarker. In some embodiments, there is provided a method of treating an individual having a mitochondrial-associated disorder comprising administering to the individual an effective amount of a composition comprising nanoparticles comprising an allosteric mTOR inhibitor (such as a limus drug) and an albumin, wherein the individual is selected for treatment based on the individual having a tissue biomarker. In some embodiments, the tissue biomarker is the presence of ragged red fibers or structurally abnormal mitochondria. In some embodiments, tissue marker is identified using immunohistochemistry. In some embodiments, tissue marker is identified using microscopy.

In some embodiments, there is provided a method of treating an individual having a mitochondrial-associated disorder comprising administering to the individual an effective amount of an allosteric mTOR inhibitor (such as a limus drug), wherein the individual is selected for treatment based on activity level of an enzyme or a coenzyme. In some embodiments, there is provided a method of treating an individual having a mitochondrial-associated disorder comprising administering to the individual an effective amount of a composition comprising nanoparticles comprising an allosteric mTOR inhibitor (such as a limus drug) and an albumin, wherein the individual is selected for treatment based on activity level of an enzyme or a coenzyme. In some embodiments, the activity level of an enzyme or a coenzyme is based on coenzyme Q10 activity, cytochrome oxidase activity, NADH dehydrogenase activity, succinate dehydrogenase activity, complex I activity, complex II activity, complex III activity, complex IV activity, complex V activity, complex I and III activity, complex II and III activity, citrate synthase activity, pyruvate dehydrogenase complex activity, tricarboxylic acid cycle enzymatic activity, or beta-oxidation enzymatic activity. In some embodiments, the activity level of an enzyme or a coenzyme is measured using a spectrophotometric assay, fluorometric assay, calorimetric assay, chemiluminescent assay, light scattering assay, microscale thermophoresis assay, radiometric assay, or chromatographic assay.

In some embodiments, there is provided a method of treating an individual having a mitochondrial-associated disorder comprising administering to the individual an effective amount of an allosteric mTOR inhibitor (such as a limus drug), wherein the individual is selected for treatment based on presence (such as a level, for example a low level) of a protein, an enzyme, or a coenzyme. In some embodiments, there is provided a method of treating an individual having a mitochondrial-associated disorder comprising administering to the individual an effective amount of a composition comprising nanoparticles comprising an allosteric mTOR inhibitor (such as a limus drug) and an albumin, wherein the individual is selected for treatment based on presence (such as a level, for example a low level) of a protein, an enzyme, or a coenzyme. In some embodiments, the enzyme or the coenzyme is selected from: coenzyme Q10, cytochrome oxidase, NADH dehydrogenase, and succinate dehydrogenase. In some embodiments, the presence of the protein, the enzyme, or the coenzyme is measured in muscle tissue. In some embodiments, the presence of the protein, the enzyme, or the coenzyme is measured by immunohistochemistry. In some embodiments, the presence of the protein, the enzyme, or the coenzyme is determined by in situ hybridization. In some embodiments, the presence of the protein, the enzyme, or the coenzyme is determined by mass spectrometry.

In some embodiments, there is provided a method of treating an individual having a mitochondrial-associated disorder comprising administering to the individual an effective amount of an allosteric mTOR inhibitor (such as a limus drug), wherein the individual is selected for treatment based on presence (such as a level, for example a low level) of one or more biomarkers. In some embodiments, there is provided a method of treating an individual having a mitochondrial-associated disorder comprising administering to the individual an effective amount of a composition comprising nanoparticles comprising an allosteric mTOR inhibitor (such as a limus drug) and an albumin, wherein the individual is selected for treatment based on presence (such as a level, for example a low level) of one or more biomarkers. In some embodiments, the biomarker or biomarkers are selected from one or more of: 3-methylglutaconate, acylcarnitine, amino acids, ammonia, carnitine, citric acid cycle intermediates, coenzyme Q10, copper, creatine, creatinine, creatinine kinase, dicarboxylic acid, electrolytes, ethylmalonate, free fatty acids, very long chain fatty acids, glucose, ketones, a lactate (such as lactic acid), myoglobin, neurotransmitters, organic acids, pyruvate, uric acid, urea, nitrogen levels, red blood cells, and white blood cells. In some embodiments, the individual is selected for treatment based on an increased ratio of lactate to pyruvate in their blood, plasma, cerebrospinal fluid, and/or urine (e.g., a sample of blood, plasma, cerebrospinal fluid, and/or urine derived from the individual). In some embodiments, the individual is selected for treatment based on a lactate to pyruvate ratio of at least 10:1 (such as at least about any of 11:1, 12:1, 13:1, 14:1, 15:1, 16:1, 17:1, 18:1, 19:1, 20:1, 25:1, 30:1, 35:1, 40:1, 45:1, 50:1, or greater). In some embodiments, the individual is selected for treatment based on a lactate to pyruvate ratio of at least 20:1. In some embodiments, the presence (such as a level) of the biomarker is detected using mass spectrometry. In some embodiments, the presence (such as a level) of the biomarker is detected using nuclear magnetic resonance.

In some embodiments, there is provided a method of treating an individual having a mitochondrial-associated disorder comprising administering to the individual an effective amount of an allosteric mTOR inhibitor (such as a limus drug), wherein the individual is selected for treatment based on having one or more symptoms. In some embodiments, there is provided a method of treating an individual having a mitochondrial-associated disorder comprising administering to the individual an effective amount of a composition comprising nanoparticles comprising an allosteric mTOR inhibitor (such as a limus drug) and an albumin, wherein the individual is selected for treatment based on having one or more symptoms. In some embodiments, the one or more symptoms is selected from: autism, learning disabilities, neurological problems, muscle weakness, loss of muscle coordination, exercise intolerance, diabetes, glucose intolerance, hypoglycemia, adrenal dysfunction, memory loss, poor growth, failure to thrive, sensory problems, development delays, drooping eyelids, paralysis of facial muscles, seizure, short stature, dementia, stroke or stroke-like episodes, hearing loss, migraines, heart muscle weakness, liver failure, renal failure, vomiting, gastrointestinal reflux, delayed gastric emptying, chronic diarrhea, and chronic constipation. In some embodiments, the one or more symptoms are assessed via a diagnostic tool selected from: audiogram, magnetic resonance imaging, computed tomography, magnetic resonance spectroscopy, electroencephalography, electrocardiography, echocardiography, and electroretinography.

In some embodiments, there is provided a method of treating an individual having a mitochondrial-associated disorder comprising administering to the individual an effective amount of an allosteric mTOR inhibitor (such as a limus drug), wherein the individual is selected for treatment based on results from a diagnostic test. In some embodiments, there is provided a method of treating an individual having a mitochondrial-associated disorder comprising administering to the individual an effective amount of a composition comprising nanoparticles comprising an allosteric mTOR inhibitor (such as a limus drug) and an albumin, wherein the individual is selected for treatment based on results from a diagnostic test. In some embodiments, the result is selected from: abnormal signal in the basal ganglia, basal ganglia calcification, cerebral atrophy, cerebellar atrophy, bilateral striatal necrosis, cerebellar hypoplasia, infarcts, and leukoencephalopathy.

In some embodiments, the sample in blood. In some embodiments, the sample is cerebrospinal fluid (CSF). In some embodiments, the sample is plasma. In some embodiments, the sample is urine.

The classification or ranking of presence (such as a level, e.g., high or low) may be determined relative to a statistical distribution of control levels. In some embodiments, the classification or ranking is relative to a control sample obtained from the individual. In some embodiment, the presence is classified or ranked relative to a statistical distribution of control samples. In some embodiments, the presence is classified or ranked relative to the presence from a control sample obtained from the individual. In some embodiments, the presence is classified or ranked relative to the lower limit of detection or quantification of an assay for measuring the presence. In some embodiments, the lack of presence is classified below the lower limit of detection or quantification of an assay for measuring the presence.

Control samples can be obtained using the same sources and methods as non-control samples. In some embodiments, the control sample is obtained from a different individual (for example an individual not having a mitochondrial-associated disorder and/or an individual sharing similar ethnic, age, and gender identity). In some embodiments, multiple control samples (for example from different individuals) are used to determine a range of levels of biomarkers in a particular tissue, organ, or cell population. In some embodiments, the control sample is a cultured tissue or cell that has been determined to be a proper control. In some embodiments, the control is a cell that does not express the biomarker. In some embodiments, the clinically accepted normal level in a standardized test is used as a control level for determining the biomarker level. In some embodiments, the reference level of biomarker in the subject is classified as high, medium or low according to a scoring system, such as an immunohistochemistry-based scoring system. In some embodiments, the reference level of biomarker in the subject is classified as a low sample when the score is less than or equal to the overall median score.

In some embodiments, the biomarker presence (such as a level) is determined by measuring the level of a biomarker in an individual and comparing to a control or reference (e.g., the median level for the given patient population or level of a second individual). For example, if the level of a biomarker for the single individual is determined to be above the median level of the patient population, that individual is determined to have a high level of the biomarker. Alternatively, if the level of a biomarker for the single individual is determined to be below the median level of the patient population, that individual is determined to have a low level of the biomarker. In some embodiments, the individual is compared to a second individual and/or a patient population which is responsive to treatment. In some embodiments, the individual is compared to a second individual and/or a patient population which is not responsive to treatment. In any of the embodiments herein, the presence (such as a level) can be determined by measuring the level of a nucleic acid encoding a biomarker. For example, if the level of an mRNA encoding a biomarker for the single individual is determined to be above the median level of the patient population, that individual is determined to have a high level of an mRNA encoding the biomarker. Alternatively, if the level of mRNA encoding the biomarker for the single individual is determined to be below the median level of the patient population, that individual is determined to have a low level of an mRNA encoding the biomarker.

In some embodiments, the reference level of a biomarker is determined by obtaining a statistical distribution of biomarker levels.

In some embodiments, bioinformatics methods are used for the determination and classification of the levels of the biomarker. Numerous alternative bioinformatics approaches have been developed to assess gene set expression profiles using gene expression profiling data. Methods include but are not limited to those described in Segal, E. et al. Nat. Genet. 34:66-176 (2003); Segal, E. et al. Nat. Genet. 36:1090-1098 (2004); Barry, W. T. et al. Bioinformatics 21:1943-1949 (2005); Tian, L. et al. Proc Nat'l Acad Sci USA 102:13544-13549 (2005); Novak B A and Jain A N. Bioinformatics 22:233-41 (2006); Maglietta R et al. Bioinformatics 23:2063-72 (2007); Bussemaker H J, BMC Bioinformatics 8 Suppl 6:S6 (2007).

In some embodiments, the presence (such as a level) of protein expression is determined, for example by immunohistochemistry. For example, the criteria for low or high levels can be made based on the number of positive staining cells and/or the intensity of the staining, for example by using an antibody that specifically recognizes the biomarker protein. In some embodiments, the level is low if less than about 1%, 5%, 10%, 15%, 20%, 25%, 30%, 35%, 40%, 45%, or 50% cells have positive staining. In some embodiments, the level is low if the staining is 1%, 5%, 10%, 15%, 20%, 25%, 30%, 35%, 40%, 45%, or 50% less intense than a positive control staining.

In some embodiments, the level is high if more than about 40%, 45%, 50%, 55%, 60%, 65%, 70%, 75%, 80%, 85%, or 90%, cells have positive staining. In some embodiments, the level is high if the staining is as intense as positive control staining. In some embodiments, the level is high if the staining is 80%, 85%, or 90% as intense as positive control staining.

In some embodiments, strong staining, moderate staining, and weak staining are calibrated levels of staining, wherein a range is established and the intensity of staining is binned within the range. In some embodiments, strong staining is staining above the 75th percentile of the intensity range, moderate staining is staining from the 25th to the 75th percentile of the intensity range, and low staining is staining below the 25th percentile of the intensity range. In some aspects one skilled in the art, and familiar with a particular staining technique, adjusts the bin size and defines the staining categories.

In some embodiments, the biomarker is evaluated from a blood sample. In some embodiments, the biomarker is evaluated from a cell-free DNA sample. In some embodiments, the biomarker is evaluated using next-generation sequencing. In some embodiments, the biomarker is evaluated using immunohistochemistry.

Further provided herein are methods of directing treatment of an individual having a mitochondrial-associated disorder by delivering a sample to a diagnostic lab for determination of biomarker levels; providing a control sample with a known level of a biomarker, wherein the level of the sample is used to provide a conclusion that a patient should receive a treatment with any one of the methods described herein.

Also provided herein are methods of directing treatment of a disease, further comprising reviewing or analyzing data relating to the presence (or level) of a biomarker in a sample; and providing a conclusion to an individual, such as a health care provider or a health care manager, about the likelihood or suitability of the individual to respond to a treatment, the conclusion being based on the review or analysis of data. In one aspect of the invention a conclusion is the transmission of the data over a network.

### Methods of Treating Metabolic Disorders and Inhibiting Cellular Glucose Consumption

The present invention provides methods of treating an individual (e.g., human) having a metabolic disorder comprising administering to the individual an effective amount of an allosteric mTOR inhibitor (such as a limus drug). In some embodiments, the invention provides methods of treating an individual (e.g., human) having a metabolic disorder comprising administering to the individual an effective amount of a composition comprising nanoparticles comprising an allosteric mTOR inhibitor and an albumin. In some embodiments, the invention provides methods of treating an individual (e.g. human) having a metabolic disorder comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin. wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus.

In some embodiments, the individual having a metabolic disorder has a disorder associated with cellular glucose consumption (e.g., abnormally high cellular glucose consumption in one or more tissues). In some embodiments, the individual having a metabolic disorder has a disorder associated with insulin resistance. In some embodiments, the individual having a metabolic disorder has hypoglycemia. In some embodiments, the individual having a metabolic disorder has hyperinsulinemic hypoglycemia. In some embodiments, the individual having a metabolic disorder has diabetes mellitus type 1. In some embodiments, the individual having a metabolic disorder has diabetes mellitus type 2. In some embodiments, the individual having a metabolic disorder has metabolic syndrome.

In some embodiments, there is provided a method of treating a disorder associated with cellular glucose consumption (e.g., abnormally high cellular glucose consumption in one or more tissues) in an individual (such as human) comprising administering to the individual an effective amount of an allosteric mTOR inhibitor (such as a limus drug). In some embodiments, the invention provides methods of treating a disorder associated with cellular glucose consumption in an individual (such as human) comprising administering to the individual an effective amount of a composition comprising nanoparticles comprising an allosteric mTOR inhibitor and an albumin. In some embodiments, the invention provides methods of treating a disorder associated with cellular glucose consumption in an individual (such as human) comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating a disorder associated with insulin resistance in an individual (such as human) comprising administering to the individual an effective amount of an allosteric mTOR inhibitor (such as a limus drug). In some embodiments, the invention provides methods of treating a disorder associated with insulin resistance in an individual (such as human) comprising administering to the individual an effective amount of a composition comprising nanoparticles comprising an allosteric mTOR inhibitor and an albumin. In some embodiments, the invention provides methods of treating a disorder associated with insulin resistance in an individual (such as human) comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating hypoglycemia in an individual (such as human) comprising administering to the individual an effective amount of an allosteric mTOR inhibitor (such as a limus drug). In some embodiments, the invention provides methods of treating hypoglycemia in an individual (such as human) comprising administering to the individual an effective amount of a composition comprising nanoparticles comprising an allosteric mTOR inhibitor and an albumin. In some embodiments, the invention provides methods of treating hypoglycemia in an individual (such as human) comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin. wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating hyperinsulinemic hypoglycemia in an individual (such as human) comprising administering to the individual an effective amount of an allosteric mTOR inhibitor (such as a limus drug). In some embodiments, the invention provides methods of treating hyperinsulinemic hypoglycemia in an individual (such as human) comprising administering to the individual an effective amount of a composition comprising nanoparticles comprising an allosteric mTOR inhibitor and an albumin. In some embodiments, the invention provides methods of treating hyperinsulinemic hypoglycemia in an individual (such as human) comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating diabetes mellitus type 1 in an individual (such as human) comprising administering to the individual an effective amount of an allosteric mTOR inhibitor (such as a limus drug). In some embodiments, the invention provides methods of treating diabetes mellitus type 1 in an individual (such as human) comprising administering to the individual an effective amount of a composition comprising nanoparticles comprising an allosteric mTOR inhibitor and an albumin. In some embodiments, the invention provides methods of treating diabetes mellitus type 1 in an individual (such as human) comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating diabetes mellitus type 2 in an individual (such as human) comprising administering to the individual an effective amount of an allosteric mTOR inhibitor (such as a limus drug). In some embodiments, the invention provides methods of treating diabetes mellitus type 2 in an individual (such as human) comprising administering to the individual an effective amount of a composition comprising nanoparticles comprising an allosteric mTOR inhibitor and an albumin. In some embodiments, the invention provides methods of treating diabetes mellitus type 2 in an individual (such as human) comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

In some embodiments, there is provided a method of treating metabolic syndrome in an individual (such as human) comprising administering to the individual an effective amount of an allosteric mTOR inhibitor (such as a limus drug). In some embodiments, the invention provides methods of treating metabolic syndrome in an individual (such as human) comprising administering to the individual an effective amount of a composition comprising nanoparticles comprising an allosteric mTOR inhibitor and an albumin. In some embodiments, the invention provides methods of treating metabolic syndrome in an individual (such as human) comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor comprises nab-sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

The present invention further provides methods of inhibiting cellular glucose consumption (e.g., abnormally high cellular glucose consumption in one or more tissues) in an individual comprising administering to the individual an effective amount of an allosteric mTOR inhibitor (such as a limus drug). In some embodiments, the invention provides methods of inhibiting cellular glucose consumption in an individual (e.g., human) comprising administering to the individual an effective amount of a composition comprising nanoparticles comprising an allosteric mTOR inhibitor and an albumin. In some embodiments, the invention provides methods of inhibiting cellular glucose consumption in an individual (e.g. human) comprising administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin, wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor comprises nab-sirolimus. In some embodiments, the allosteric mTOR inhibitor is nab-sirolimus.

In some embodiments, there is provided a method of reducing cellular glucose consumption (e.g., abnormally high cellular glucose consumption in one or more tissues) in an individual comprising administering to the individual an effective amount of an allosteric mTOR inhibitor (such as a limus drug). In some embodiments, the method comprises administering to the individual an effective amount of a composition comprising nanoparticles comprising an allosteric mTOR inhibitor and an albumin. In some embodiments, the method comprises administering to the individual an effective amount of a composition comprising nanoparticles comprising a limus drug and an albumin. In some embodiments, the method comprises administering to the individual an effective amount of a composition comprising nanoparticles comprising a limus drug and an albumin, wherein the limus drug in the nanoparticles is associated (e.g., coated) with the albumin. In some embodiments, the method comprises administering to the individual an effective amount of a composition comprising nanoparticles comprising a limus drug and an albumin, wherein the nanoparticles have an average particle size of no greater than about 150 nm (such as no greater than about 120 nm). In some embodiments, the average or mean diameter of the nanoparticles is about 10 nm to about 150 nm. In some embodiments, the average or mean diameter of the nanoparticles is about 40 nm to about 120 nm. In some embodiments, the method comprises administering to the individual an effective amount of a composition comprising nanoparticles comprising a limus drug and an albumin. wherein the nanoparticles comprise a limus drug associated (e.g., coated) with albumin, wherein the nanoparticles have an average particle size of no greater than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the method comprises administering to the individual an effective amount of a composition comprising nanoparticles comprising a limus drug and an albumin, wherein the weight ratio of albumin and limus drug in the nanoparticle composition is about 1:1 to about 11:1 (such as about 1:1 to about 9:1). In some embodiments, the method comprises administering to the individual an effective amount of a composition comprising nanoparticles comprising sirolimus and human albumin, wherein the nanoparticles comprise sirolimus associated (e.g., coated) with human albumin, wherein the nanoparticles have an average particle size of no greater than about 150 nm (such as no greater than about 120 nm, for example about 100 nm), wherein the weight ratio of human albumin and sirolimus in the nanoparticle composition is about 1:1 to about 9:1 (such as about 8:1, about 8.5:1, or about 9:1). In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m- to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor comprises nab-sirolimus. In some embodiments, the allosteric mTOR inhibitor is nab-sirolimus. In some embodiments, the individual has abnormally high cellular glucose consumption in a tissue, and cellular glucose consumption in the tissue is reduced.

### Methods of Treating Additional Diseases

Further provided are methods of treating an individual having a disease, such as fetal dilated cardiomyopathy. In some embodiments, there is provided a method of treating fetal dilated cardiomyopathy in an individual comprising administering to the individual an effective amount of an allosteric mTOR inhibitor, such as an effective amount of a composition comprising nanoparticles comprising an allosteric mTOR inhibitor and an albumin. In some embodiments, the allosteric mTOR inhibitor is a limus drug, such as a composition comprising a limus drug and an albumin. In some embodiments, the allosteric mTOR inhibitor is sirolimus. In some embodiments, the sirolimus is in a composition comprising nanoparticles comprising sirolimus and an albumin. In some embodiments, the albumin is human albumin (such as human serum albumin). In some embodiments, the nanoparticles comprise sirolimus associated (e.g., coated) with albumin. In some embodiments, the average particle size of the nanoparticles in a nanoparticle composition is no more than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the sirolimus is in a composition comprising an albumin stabilized nanoparticle formulation of sirolimus. In some embodiments, the allosteric mTOR inhibitor is nab-sirolimus.

Further provided are methods of treating an individual having a disease, such as tuberous sclerosis complex (TSC) and related disorders, including dilated cardiomyopathy in TSC. In some embodiments, there is provided a method of treating tuberous sclerosis, including dilated cardiomyopathy in TSC, in an individual comprising administering to the individual an effective amount of an allosteric mTOR inhibitor, such as an effective amount of a composition comprising nanoparticles comprising an allosteric mTOR inhibitor and an albumin. In some embodiments, the allosteric mTOR inhibitor is a limus drug, such as a composition comprising a limus drug and an albumin. In some embodiments, the allosteric mTOR inhibitor is sirolimus. In some embodiments, the sirolimus is in a composition comprising nanoparticles comprising sirolimus and an albumin. In some embodiments, the albumin is human albumin (such as human serum albumin). In some embodiments, the nanoparticles comprise sirolimus associated (e.g., coated) with albumin. In some embodiments, the average particle size of the nanoparticles in a nanoparticle composition is no more than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the sirolimus is in a composition comprising an albumin stabilized nanoparticle formulation of sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

Further provided are methods of treating an individual having a disease, such as childhood onset cardiomyopathy. In some embodiments, there is provided a method of treating childhood onset cardiomyopathy in an individual comprising administering to the individual an effective amount of an allosteric mTOR inhibitor, such as an effective amount of a composition comprising nanoparticles comprising an allosteric mTOR inhibitor and an albumin. In some embodiments, the allosteric mTOR inhibitor is a limus drug, such as a composition comprising a limus drug and an albumin. In some embodiments, the allosteric mTOR inhibitor is sirolimus. In some embodiments, the sirolimus is in a composition comprising nanoparticles comprising sirolimus and an albumin. In some embodiments, the albumin is human albumin (such as human serum albumin). In some embodiments, the nanoparticles comprise sirolimus associated (e.g., coated) with albumin. In some embodiments, the average particle size of the nanoparticles in a nanoparticle composition is no more than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the sirolimus is in a composition comprising an albumin stabilized nanoparticle formulation of sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

Further provided are methods of treating an individual having a disease, such as Noonan syndrome. In some embodiments, there is provided a method of treating Noonan syndrome in an individual comprising administering to the individual an effective amount of an allosteric mTOR inhibitor, such as an effective amount of a composition comprising nanoparticles comprising an allosteric mTOR inhibitor and an albumin. In some embodiments, the allosteric mTOR inhibitor is a limus drug, such as a composition comprising a limus drug and an albumin. In some embodiments, the allosteric mTOR inhibitor is sirolimus. In some embodiments, the sirolimus is in a composition comprising nanoparticles comprising sirolimus and an albumin. In some embodiments, the albumin is human albumin (such as human serum albumin). In some embodiments, the nanoparticles comprise sirolimus associated (e.g., coated) with albumin. In some embodiments, the average particle size of the nanoparticles in a nanoparticle composition is no more than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the sirolimus is in a composition comprising an albumin stabilized nanoparticle formulation of sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

Further provided are methods of treating an individual having a disease, such as polycystic kidney disease. In some embodiments, there is provided a method of treating polycystic kidney disease in an individual comprising administering to the individual an effective amount of an allosteric mTOR inhibitor, such as an effective amount of a composition comprising nanoparticles comprising an allosteric mTOR inhibitor and an albumin. In some embodiments, the allosteric mTOR inhibitor is a limus drug, such as a composition comprising a limus drug and an albumin. In some embodiments, the allosteric mTOR inhibitor is sirolimus. In some embodiments, the sirolimus is in a composition comprising nanoparticles comprising sirolimus and an albumin. In some embodiments, the albumin is human albumin (such as human serum albumin). In some embodiments, the nanoparticles comprise sirolimus associated (e.g., coated) with albumin. In some embodiments, the average particle size of the nanoparticles in a nanoparticle composition is no more than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the sirolimus is in a composition comprising an albumin stabilized nanoparticle formulation of sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

Further provided are methods of treating an individual having a disease, such as age-related and genetically induced hypertrophic cardiomyopathy. In some embodiments, there is provided a method of treating age-related and genetically induced hypertrophic cardiomyopathy in an individual comprising administering to the individual an effective amount of an allosteric mTOR inhibitor, such as an effective amount of a composition comprising nanoparticles comprising an allosteric mTOR inhibitor and an albumin. In some embodiments, the allosteric mTOR inhibitor is a limus drug, such as a composition comprising a limus drug and an albumin. In some embodiments, the allosteric mTOR inhibitor is sirolimus. In some embodiments, the sirolimus is in a composition comprising nanoparticles comprising sirolimus and an albumin. In some embodiments, the albumin is human albumin (such as human serum albumin). In some embodiments, the nanoparticles comprise sirolimus associated (e.g., coated) with albumin. In some embodiments, the average particle size of the nanoparticles in a nanoparticle composition is no more than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the sirolimus is in a composition comprising an albumin stabilized nanoparticle formulation of sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

Further provided are methods of treating an individual having a disease, such as a rheumatic disease. In some embodiments, there is provided a method of treating a rheumatic disease in an individual comprising administering to the individual an effective amount of an allosteric mTOR inhibitor, such as an effective amount of a composition comprising nanoparticles comprising an allosteric mTOR inhibitor and an albumin. In some embodiments, the allosteric mTOR inhibitor is a limus drug, such as a composition comprising a limus drug and an albumin. In some embodiments, the allosteric mTOR inhibitor is sirolimus. In some embodiments, the sirolimus is in a composition comprising nanoparticles comprising sirolimus and an albumin. In some embodiments, the albumin is human albumin (such as human serum albumin). In some embodiments, the nanoparticles comprise sirolimus associated (e.g., coated) with albumin. In some embodiments, the average particle size of the nanoparticles in a nanoparticle composition is no more than about 150 nm (such as no greater than about 120 nm). In some embodiments, the weight ratio of albumin and allosteric mTOR inhibitor in the nanoparticle composition is about 1:1 to about 11:1 (such as about 1:1 to about 9:1). In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1. In some embodiments, the weight ratio of albumin and allosteric mTOR inhibitor in the nanoparticle composition is about 10:1. In some embodiments, the weight ratio of albumin and allosteric mTOR inhibitor in the nanoparticle composition is about 11:1. In some embodiments, the sirolimus is in a composition comprising an albumin stabilized nanoparticle formulation of sirolimus. In some embodiments, the allosteric mTOR inhibitor is nab-sirolimus. In some embodiments, the allosteric mTOR inhibitor (such as a nanoparticle limus drug) is administered at a dose of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²). In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor is administered weekly. In some embodiments, the allosteric mTOR inhibitor is administered once every two weeks. In some embodiments, the allosteric mTOR inhibitor is administered once every three weeks. In some embodiments, the allosteric mTOR inhibitor is administered once every four weeks. In some embodiments, the allosteric mTOR inhibitor is administered daily. In some embodiments, the allosteric mTOR inhibitor is administered once every three days.

Allosteric mTOR Inhibitor

The methods described herein, in some embodiments, comprise administration of an allosteric mTOR inhibitor. Allosteric mTOR inhibitors do not inhibit mTOR via binding to the ATP catalytic site of mTOR. mTOR is a serine/threonine-specific protein kinase downstream of the phosphatidylinositol 3-kinase (PI3K)/Akt (protein kinase B) pathway, and a key regulator of cell survival, proliferation, stress, and metabolism.

The mammalian target of rapamycin (mTOR) (also known as mechanistic target of rapamycin or FK506 binding protein 12-rapamycin associated protein 1 (FRAP1)) is an atypical serine/threonine protein kinase that is present in two distinct complexes, mTOR Complex 1 (mTORC1) and mTOR Complex 2 (mTORC2). mTORC1 is composed of mTOR, regulatory-associated protein of mTOR (Raptor), mammalian lethal with SEC 13 protein 8 (MLST8), PRAS40 and DEPTOR (Kim et al. (2002) Cell 110: 163-75; Fang et al. (2001) Science 294 (5548): 1942-5). mTORC1 integrates four major signal inputs: nutrients (such as amino acids and phosphatidic acid), growth factors (insulin), energy and stress (such as hypoxia and DNA damage). Amino acid availability is signaled to mTORC1 via a pathway involving the Rag and Ragulator (LAMTOR1-3) Growth factors and hormones (e.g. insulin) signal to mTORC1 via Akt, which inactivates TSC2 to prevent inhibition of mTORC1. Alternatively, low ATP levels lead to the AMPK-dependent activation of TSC2 and phosphorylation of raptor to reduce mTORC1 signaling proteins.

Active mTORC1 has a number of downstream biological effects including translation of mRNA via the phosphorylation of downstream targets (4E-BPI and p70 S6 Kinase), suppression of autophagy (Atg13, ULK1), ribosome biogenesis, and activation of transcription leading to mitochondrial metabolism or adipogenesis. Accordingly, mTORC1 activity promotes either cellular growth when conditions are favorable or catabolic processes during stress or when conditions are unfavorable.

mTORC2 is composed of mTOR, rapamycin-insensitive companion of mTOR (RICTOR), GβL, and mammalian stress-activated protein kinase interacting protein 1 (mSIN1). In contrast to mTORC1, for which many upstream signals and cellular functions have been defined (see above), relatively little is known about mTORC2 biology. mTORC2 regulates cytoskeletal organization through its stimulation of F-actin stress fibers, paxillin, RhoA, Rac1, Cdc42, and protein kinase C α (PKCα). It had been observed that knocking down mTORC2 components affects actin polymerization and perturbs cell morphology (Jacinto et al. (2004) Nat. Cell Biol. 6, 1122-1128; Sarbassov et al. (2004) Curr. Biol. 14, 1296-1302). This suggests that mTORC2 controls the actin cytoskeleton by promoting protein kinase Cα (PKCα) phosphorylation, phosphorylation of paxillin and its relocalization to focal adhesions, and the GTP loading of RhoA and Rac1. The molecular mechanism by which mTORC2 regulates these processes has not been determined.

In some embodiments, the allosteric mTOR inhibitor is an inhibitor of mTORC1. In some embodiments, the allosteric mTOR inhibitor is an inhibitor of mTORC2.

In some embodiments, the allosteric mTOR inhibitor is a limus drug, which includes sirolimus and its analogues. Examples of limus drugs include, but are not limited to, temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the limus drug is selected from the group consisting of temsirolimus (CCI-779), everolimus (RAD001), ridaforolimus (AP-23573), deforolimus (MK-8669), zotarolimus (ABT-578), pimecrolimus, and tacrolimus (FK-506).

In some embodiments, the allosteric mTOR inhibitor is sirolimus. Sirolimus is macrolide antibiotic that complexes with FKBP-12 and inhibits the mTOR pathway by binding mTORC1.

In some embodiments, the allosteric mTOR inhibitor is selected from the group consisting of sirolimus (rapamycin), everolimus (also known as RAD001, Zortress, Certican, and Afinitor), temsirolimus (also known as CCI-779 and Torisel). Ku-0063794, Palomid 529, and deforolimus (also known as ridaforolimus).

Everolimus is the 40-O-(2-hydroxyethyl) derivative of rapamycin and binds the cyclophilin FKBP-12, and this complex also mTORC1. Temsirolimus is a prodrug of rapamycin that forms a complex with the FK506-binding protein and prohibits the activation of mTOR when it resides in the mTORC1 complex. KU-0063794 is a small molecule that inhibits the phosphorylation of mTORC1 at Ser2448 in a dose-dependent and time-dependent manner. Palomid 529 is a small molecule inhibitor of mTORC1 that lacks affinity for ABCB1/ABCG2 and has good brain penetration (Lin et al. (2013) Int J Cancer DOI: 10.1002/ijc.28126 (e-published ahead of print). Deforolimus (Ridaforolimus, AP23573, MK-8669) is a selective allosteric mTOR inhibitor.

### Nanoparticle Compositions

The methods described herein, in some embodiments, comprise administration of an allosteric mTOR inhibitor, wherein the allosteric mTOR inhibitor comprises a composition comprising nanoparticles comprising an allosteric mTOR inhibitor and an albumin.

Nanoparticles of poorly water soluble drugs have been disclosed in, for example, U.S. Pat. Nos. 5,916,596; 6,506,405; 6,749,868, 6,537,579, 7,820,788, and also in U.S. Publication Nos.: 20060263434; and 2007/0082838; and PCT Patent Application WO08/137148, each of which is incorporated by reference in their entirety.

In some embodiments, the nanoparticle composition comprises nanoparticles with an average or mean diameter of no greater than about 1000 nanometers (nm), such as no greater than about (or less than about) any of 900, 800, 700, 600, 500, 400, 300, 200, 150, 120, and 100 nm. In some embodiments, the average or mean diameters of the nanoparticles is no greater than about 150 nm (such as no greater than about 120 nm). In some embodiments, the average or mean diameters of the nanoparticles is no greater than about 120 nm. In some embodiments, the average or mean diameter of the nanoparticles is about 10 nm to about 150 nm. In some embodiments, the average or mean diameter of the nanoparticles is about 40 nm to about 120 nm. In some embodiments, the nanoparticles are sterile-filterable.

In some embodiments, the nanoparticles in the nanoparticle compositions described herein have an average diameter of no greater than about 150 nm, including for example no greater than about any one of 140, 130, 120, 110, 100, 90, 80, 70, or 60 nm. In some embodiments, at least about 50% (for example at least about any one of 60%, 70%, 80%, 90%, 95%, or 99%) of the nanoparticles in the nanoparticle compositions have a diameter of no greater than about 150 nm, including for example no greater than about any one of 140, 130, 120, 110, 100, 90, 80, 70, or 60 nm. In some embodiments, at least about 50% (for example at least any one of 60%, 70%, 80%, 90%, 95%, or 99%) of the nanoparticles in the nanoparticle compositions fall within the range of about 20 nm to about 150 nm, including for example about 40 nm to about 120 nm.

In some embodiments, the albumin has sulfhydryl groups that can form disulfide bonds. In some embodiments, at least about 5% (including for example at least about any one of 10%, 15%, 20%, 25%, 30%, 40%, 50%, 60%, 70%, 80%, or 90%) of the albumin in the nanoparticle portion of the nanoparticle composition are crosslinked (for example crosslinked through one or more disulfide bonds).

In some embodiments, the nanoparticles comprising the allosteric mTOR inhibitor (such as a limus drug, e.g., sirolimus) are associated (e.g., coated) with an albumin (e.g., human albumin or human serum albumin). In some embodiments, the nanoparticle composition comprises an allosteric mTOR inhibitor (such as a limus drug, for example sirolimus) in both nanoparticle and non-nanoparticle forms (e.g., in the form of solutions or in the form of soluble albumin/nanoparticle complexes), wherein at least about any one of 50%, 60%, 70%, 80%, 90%, 95%, or 99% of the allosteric mTOR inhibitor (such as a limus drug, e.g., sirolimus) in the nanoparticle composition are in nanoparticle form. In some embodiments, the allosteric mTOR inhibitor (such as a limus drug, e.g., sirolimus) in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of an allosteric mTOR inhibitor (such as a limus drug, for example sirolimus) that is substantially free of polymeric materials (such as polymeric matrix).

In some embodiments, the nanoparticle composition comprises an albumin in both nanoparticle and non-nanoparticle portions of the nanoparticle composition, wherein at least about any one of 50%, 60%, 70%, 80%, 90%, 95%, or 99% of the albumin in the nanoparticle composition are in non-nanoparticle portion of the nanoparticle composition.

In some embodiments, the weight ratio of an albumin (such as human albumin or human serum albumin) and an allosteric mTOR inhibitor in the nanoparticle composition is about 18:1 or less, such as about 15:1 or less, for example about 10:1 or less. In some embodiments, the weight ratio of an albumin (such as human albumin or human serum albumin) and an allosteric mTOR inhibitor (such as a limus drug, for example sirolimus) in the nanoparticle composition falls within the range of any one of about 1:1 to about 18:1, about 2:1 to about 15:1, about 3:1 to about 13:1, about 4:1 to about 12:1, about 5:1 to about 10:1. In some embodiments, the weight ratio of an albumin and an allosteric mTOR inhibitor (such as a limus drug, for example sirolimus) in the nanoparticle portion of the nanoparticle composition is about any one of 1:2, 1:3, 1:4, 1:5, 1:9, 1:10, 1:15, or less. In some embodiments, the weight ratio of the albumin (such as human albumin or human serum albumin) and the allosteric mTOR inhibitor (such as a limus drug, e.g., sirolimus) in the nanoparticle composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of albumin and allosteric mTOR inhibitor in the nanoparticle composition is about 8:1. In some embodiments, the weight ratio of albumin and allosteric mTOR inhibitor in the nanoparticle composition is about 8.5:1. In some embodiments, the weight ratio of albumin and allosteric mTOR inhibitor in the nanoparticle composition is about 9:1.

In some embodiments, the nanoparticle composition comprises one or more of the above characteristics.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulfide bridges, one free thiol (Cys 34), and a single tryptophan (Trp 214). Intravenous use of HSA solution has been indicated for the prevention and treatment of hypovolumic shock (see, e.g., Tullis, JAMA, 237: 355-360, 460-463, (1977)) and Houser et al., Surgery, Gynecology and Obstetrics, 150: 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary (including domestic pets and agricultural context). Human serum albumin (HSA) has multiple hydrophobic binding sites (a total of eight for fatty acids, an endogenous ligand of HSA) and binds a diverse set of drugs, especially neutral and negatively charged hydrophobic compounds (Goodman et al., The Pharmacological Basis of Therapeutics, 9th ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (199b), and Carter et al., Adv. Protein. Chem., 45, 153-203 (1994)). Sirolimus and propofol have been shown to bind HSA (see, e.g., Paal et al., Eur. J. Biochem., 268(7), 2187-91 (200a), Purcell et al., Biochim. Biophys. Acta, 1478(a), 61-8 (2000), Altmayer et al., Arzneimittelforschung, 45, 1053-6 (1995), and Garrido et al., Rev. Esp. Anestestiol. Reanim., 41, 308-12 (1994)). In addition, docetaxel has been shown to bind to human plasma proteins (see. e.g., Urien et al., Invest. New Drugs, 14(b), 147-51 (1996)).

The albumin in the nanoparticle composition, in part, makes the allosteric mTOR inhibitor (such as a limus drug, e.g., sirolimus) more readily suspendable in an aqueous medium or helps maintain the suspension as compared to compositions not comprising an albumin. This can avoid the use of toxic solvents (or surfactants) for solubilizing the allosteric mTOR inhibitor, and thereby can reduce one or more side effects of administration of the allosteric mTOR inhibitor (such as a limus drug, e.g., sirolimus) into an individual (such as a human). Thus, in some embodiments, the nanoparticle composition described herein is substantially free (such as free) of surfactants, such as Cremophor (or polyoxyethylated castor oil, including Cremophor EL® (BASF)). In some embodiments, the nanoparticle composition is substantially free (such as free) of surfactants. A composition is “substantially free of Cremophor” or “substantially free of surfactant” if the amount of Cremophor or surfactant in the nanoparticle composition is not sufficient to cause one or more side effect(s) in an individual when the nanoparticle composition is administered to the individual. In some embodiments, the nanoparticle composition contains less than about any one of 20%, 15%, 10%, 7.5%, 5%, 2.5%, or 1% organic solvent or surfactant. In some embodiments, the albumin is human albumin or human serum albumin. In some embodiments, the albumin is recombinant albumin.

The amount of an albumin in the nanoparticle composition described herein will vary depending on other components in the nanoparticle composition. In some embodiments, the nanoparticle composition comprises an albumin in an amount that is sufficient to stabilize the allosteric mTOR inhibitor (such as a limus drug. e.g., sirolimus) in an aqueous suspension, for example, in the form of a stable colloidal suspension (such as a stable suspension of nanoparticles). In some embodiments, the albumin is in an amount that reduces the sedimentation rate of the allosteric mTOR inhibitor (such as a limus drug, e.g., sirolimus) in an aqueous medium. For particle-containing compositions, the amount of the the albumin also depends on the size and density of nanoparticles of the allosteric mTOR inhibitor.

An allosteric mTOR inhibitor (such as a limus drug, for example sirolimus) is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as human). Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed under the optical microscope at 1000 times, at about fifteen minutes after preparation of the suspension. Stability can also be evaluated under accelerated testing conditions, such as at a temperature that is higher than about 40° C.

In some embodiments, the albumin is present in an amount that is sufficient to stabilize the allosteric mTOR inhibitor (such as a limus drug, e.g., sirolimus) in an aqueous suspension at a certain concentration. For example, the concentration of the allosteric mTOR inhibitor (such as a limus drug. e.g., sirolimus) in the nanoparticle composition is about 0.1 to about 100 mg/ml, including for example any of about 0.1 to about 50 mg/ml, about 0.1 to about 20 mg/ml, about 1 to about 10 mg/ml, about 2 mg/ml to about 8 mg/ml, about 4 to about 6 mg/ml, or about 5 mg/ml. In some embodiments, the concentration of the allosteric mTOR inhibitor (such as a limus drug, e.g., sirolimus) is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the nanoparticle composition is free or substantially free of surfactant (such as Cremophor).

In some embodiments, the nanoparticle composition, in liquid form, comprises from about 0.1% to about 50% (w/v) (e.g. about 0.5% (w/v), about 5% (w/v), about 10% (w/v), about 15% (w/v), about 20% (w/v), about 30% (w/v), about 40% (w/v), or about 50% (w/v)) of an albumin. In some embodiments, the nanoparticle composition, in liquid form, comprises about 0.5% to about 5% (w/v) of an albumin.

In some embodiments, the weight ratio of albumin to the allosteric mTOR inhibitor (such as a limus drug, e.g., sirolimus) in the nanoparticle composition is such that a sufficient amount of allosteric mTOR inhibitor binds to, or is transported into, the cell. While the weight ratio of an albumin to allosteric mTOR inhibitor will have to be optimized for different albumins and allosteric mTOR inhibitor combinations, generally the weight ratio of an albumin, to allosteric mTOR inhibitor (such as a limus drug. e.g., sirolimus) (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the albumin to allosteric mTOR inhibitor weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human albumin or human serum albumin) to the allosteric mTOR inhibitor in the nanoparticle composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the albumin allows the nanoparticle composition to be administered to an individual (such as human) without significant side effects. In some embodiments, the albumin (such as human serum albumin or human albumin) is in an amount that is effective to reduce one or more side effects of administration of the allosteric mTOR inhibitor (such as a limus drug, e.g., sirolimus) to a human. The term “reducing one or more side effects” of administration of the allosteric mTOR inhibitor (such as a limus drug, e.g., sirolimus) refers to reduction, alleviation, elimination, or avoidance of one or more undesirable effects caused by the allosteric mTOR inhibitor, as well as side effects caused by delivery vehicles (such as solvents that render the limus drugs suitable for injection) used to deliver the allosteric mTOR inhibitor. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with limus drugs (such as sirolimus) can be reduced.

In some embodiments, the nanoparticle compositions described herein comprise nanoparticles comprising an allosteric mTOR inhibitor (such as a limus drug, for example sirolimus) and an albumin (such as human albumin or human serum albumin), wherein the nanoparticles have an average diameter of no greater than about 150 nm. In some embodiments, the nanoparticle compositions described herein comprise nanoparticles comprising an allosteric mTOR inhibitor (such as a limus drug, for example sirolimus) and an albumin (such as human albumin or human serum albumin), wherein the nanoparticles have an average diameter of no greater than about 150 nm. In some embodiments, the nanoparticle compositions described herein comprise nanoparticles comprising an allosteric mTOR inhibitor (such as a limus drug, for example sirolimus) and an albumin (such as human albumin or human serum albumin), wherein the nanoparticles have an average diameter of no greater than about 150 nm (for example about 100 nm). In some embodiments, the nanoparticle compositions described herein comprise nanoparticles comprising sirolimus and human albumin (such as human serum albumin), wherein the nanoparticles have an average diameter of no greater than about 150 nm (for example about 100 nm).

In some embodiments, the nanoparticle compositions described herein comprise nanoparticles comprising an allosteric mTOR inhibitor (such as a limus drug, for example sirolimus) and an albumin (such as human albumin or human serum albumin), wherein the nanoparticles have an average diameter of no greater than about 150 nm, wherein the weight ratio of the albumin and the allosteric mTOR inhibitor in the nanoparticle composition is no greater than about 9:1 (such as about 9:1, about 8.5:1, or about 8:1). In some embodiments, the nanoparticle compositions described herein comprise nanoparticles comprising an allosteric mTOR inhibitor (such as a limus drug, for example sirolimus) and an albumin (such as human albumin or human serum albumin), wherein the nanoparticles have an average diameter of no greater than about 150 nm, wherein the weight ratio of the albumin and the allosteric mTOR inhibitor in the nanoparticle composition is no greater than about 9:1 (such as about 9:1, about 8.5:1, or about 8:1). In some embodiments, the nanoparticle compositions described herein comprise nanoparticles comprising an allosteric mTOR inhibitor (such as a limus drug, for example sirolimus) and an albumin (such as human albumin or human serum albumin), wherein the nanoparticles have an average diameter of about 150 nm, wherein the weight ratio of the albumin and the allosteric mTOR inhibitor in the nanoparticle composition is no greater than about 9:1 (such as about 9:1, about 8.5:1, or about 8:1). In some embodiments, the nanoparticle compositions described herein comprise nanoparticles comprising sirolimus and human albumin (such as human serum albumin), wherein the nanoparticles have an average diameter of no greater than about 150 nm (for example about 100 nm), wherein the weight ratio of albumin and sirolimus inhibitor in the nanoparticle composition is about 9:1 or about 8:1.

In some embodiments, the nanoparticle compositions described herein comprise nanoparticles comprising an allosteric mTOR inhibitor (such as a limus drug, for example sirolimus) associated (e.g., coated) with an albumin (such as human albumin or human serum albumin). In some embodiments, the nanoparticle compositions described herein comprise nanoparticles comprising an allosteric mTOR inhibitor (such as a limus drug, for example sirolimus) associated (e.g., coated) with an albumin (such as human albumin or human serum albumin), wherein the nanoparticles have an average diameter of no greater than about 150 nm. In some embodiments, the nanoparticle compositions described herein comprise nanoparticles comprising an allosteric mTOR inhibitor (such as a limus drug, for example sirolimus) associated (e.g., coated) with an albumin (such as human albumin or human serum albumin), wherein the nanoparticles have an average diameter of no greater than about 150 nm. In some embodiments, the nanoparticle compositions described herein comprise nanoparticles comprising an allosteric mTOR inhibitor (such as a limus drug, for example sirolimus) associated (e.g., coated) with an albumin (such as human albumin or human serum albumin), wherein the nanoparticles have an average diameter of no greater than about 150 nm (for example about 100 nm). In some embodiments, the nanoparticle compositions described herein comprise nanoparticles comprising sirolimus associated (e.g., coated) with human albumin (such as human serum albumin), wherein the nanoparticles have an average diameter of no greater than about 150 nm (for example about 100 nm).

In some embodiments, the nanoparticle compositions described herein comprise nanoparticles comprising an allosteric mTOR inhibitor (such as a limus drug, for example sirolimus) associated (e.g., coated) with an albumin (such as human albumin or human serum albumin), wherein the weight ratio of the albumin and the allosteric mTOR inhibitor in the nanoparticle composition is no greater than about 9:1 (such as about 9:1, about 8.5:1, or about 8:1). In some embodiments, the nanoparticle compositions described herein comprise nanoparticles comprising an allosteric mTOR inhibitor (such as a limus drug, for example sirolimus) associated (e.g., coated) with an albumin (such as human albumin or human serum albumin), wherein the nanoparticles have an average diameter of no greater than about 150 nm, wherein the weight ratio of the albumin and the allosteric mTOR inhibitor in the nanoparticle composition is no greater than about 9:1 (such as about 9:1, about 8.5:1, or about 8:1). In some embodiments, the nanoparticle compositions described herein comprise nanoparticles comprising an allosteric mTOR inhibitor (such as a limus drug, for example sirolimus) associated (e.g., coated) with an albumin (such as human albumin or human serum albumin), wherein the nanoparticles have an average diameter of no greater than about 150 nm, wherein the weight ratio of the albumin and the allosteric mTOR inhibitor in the nanoparticle composition is no greater than about 9:1 (such as about 9:1, about 8.5:1, or about 8:1). In some embodiments, the nanoparticle compositions described herein comprise nanoparticles comprising an allosteric mTOR inhibitor (such as a limus drug, for example sirolimus) associated (e.g., coated) with an albumin (such as human albumin or human serum albumin), wherein the nanoparticles have an average diameter of about 150 nm, wherein the weight ratio of the albumin and the allosteric mTOR inhibitor in the nanoparticle composition is no greater than about 9:1 (such as about 9:1, about 8.5:1, or about 8:1). In some embodiments, the nanoparticle compositions described herein comprise nanoparticles comprising sirolimus associated (e.g., coated) with human albumin (such as human serum albumin), wherein the nanoparticles have an average diameter of no greater than about 150 nm (for example about 100 nm), wherein the weight ratio of albumin and the sirolimus in the nanoparticle composition is about 9:1 or about 8:1.

In some embodiments, the nanoparticle compositions described herein comprise nanoparticles comprising an allosteric mTOR inhibitor (such as a limus drug, for example sirolimus) stabilized by an albumin (such as human albumin or human serum albumin). In some embodiments, the nanoparticle compositions described herein comprise nanoparticles comprising an allosteric mTOR inhibitor (such as a limus drug, for example sirolimus) stabilized by an albumin (such as human albumin or human serum albumin), wherein the nanoparticles have an average diameter of no greater than about 150 nm. In some embodiments, the nanoparticle compositions described herein comprise nanoparticles comprising an allosteric mTOR inhibitor (such as a limus drug, for example sirolimus) stabilized by an albumin (such as human albumin or human serum albumin), wherein the nanoparticles have an average diameter of no greater than about 150 nm. In some embodiments, the nanoparticle compositions described herein comprise nanoparticles comprising an allosteric mTOR inhibitor (such as a limus drug, for example sirolimus) stabilized by an albumin (such as human albumin or human serum albumin), wherein the nanoparticles have an average diameter of no greater than about 150 nm (for example about 100 nm). In some embodiments, the nanoparticle compositions described herein comprise nanoparticles comprising sirolimus stabilized by human albumin (such as human serum albumin), wherein the nanoparticles have an average diameter of no greater than about 150 nm (for example about 100 nm).

In some embodiments, the nanoparticle compositions described herein comprise nanoparticles comprising an allosteric mTOR inhibitor (such as a limus drug, for example sirolimus) stabilized by an albumin (such as human albumin or human serum albumin), wherein the weight ratio of the albumin and the allosteric mTOR inhibitor in the nanoparticle composition is no greater than about 9:1 (such as about 9:1, about 8.5:1, or about 8:1). In some embodiments, the nanoparticle compositions described herein comprise nanoparticles comprising an allosteric mTOR inhibitor (such as a limus drug, for example sirolimus) stabilized by an albumin (such as human albumin or human serum albumin), wherein the nanoparticles have an average diameter of no greater than about 150 nm, wherein the weight ratio of the albumin and the allosteric mTOR inhibitor in the nanoparticle composition is no greater than about 9:1 (such as about 9:1, about 8.5:1, or about 8:1). In some embodiments, the nanoparticle compositions described herein comprise nanoparticles comprising an allosteric mTOR inhibitor (such as a limus drug, for example sirolimus) stabilized by an albumin (such as human albumin or human serum albumin), wherein the nanoparticles have an average diameter of no greater than about 150 nm, wherein the weight ratio of the albumin and the allosteric mTOR inhibitor in the nanoparticle composition is no greater than about 9:1 (such as about 9:1, about 8.5:1, or about 8:1). In some embodiments, the nanoparticle compositions described herein comprise nanoparticles comprising an allosteric mTOR inhibitor (such as a limus drug, for example sirolimus) stabilized by an albumin (such as human albumin or human serum albumin), wherein the nanoparticles have an average diameter of about 150 nm, wherein the weight ratio of the albumin and the allosteric mTOR inhibitor in the nanoparticle composition is no greater than about 9:1 (such as about 9:1, about 8.5:1, or about 8:1). In some embodiments, the nanoparticle compositions described herein comprise nanoparticles comprising sirolimus stabilized by human albumin (such as human serum albumin), wherein the nanoparticles have an average diameter of no greater than about 150 nm (for example about 100 nm), wherein the weight ratio of albumin and the sirolimus in the nanoparticle composition is about 9:1 or about 8:1.

In some embodiments, the nanoparticle composition comprises nab-sirolimus. In some embodiments, the nanoparticle composition is nab-sirolimus. Nab-sirolimus is a formulation of sirolimus stabilized by human albumin USP, which can be dispersed in directly injectable physiological solution. The weight ratio of human albumin and sirolimus is about 8:1 to about 9:1. When dispersed in a suitable aqueous medium such as 0.9% sodium chloride injection or 5% dextrose injection, nab-sirolimus forms a stable colloidal suspension of sirolimus. The mean particle size of the nanoparticles in the colloidal suspension is about 100 nanometers. Since HSA is freely soluble in water, nab-sirolimus can be reconstituted in a wide range of concentrations ranging from dilute (0.1 mg/ml sirolimus) to concentrated (20 mg/ml sirolimus), including for example about 2 mg/ml to about 8 mg/ml, or about 5 mg/ml.

Methods of making nanoparticle compositions are known in the art. For example, nanoparticles containing allosteric mTOR inhibitor (such as a limus drug, e.g., sirolimus) and an albumin (such as human serum albumin or human albumin) can be prepared under conditions of high shear forces (e.g., sonication, high pressure homogenization, or the like). These methods are disclosed in, for example, U.S. Pat. Nos. 5,916,596; 6,506,405; 6,749,868, 6,537,579 and 7,820,788 and also in U.S. Pat. Pub. Nos. 2007/0082838, 2006/0263434 and PCT Application WO08/137148.

Briefly, the allosteric mTOR inhibitor (such as a limus drug, e.g., sirolimus) is dissolved in an organic solvent, and the solution can be added to an albumin solution. The mixture is subjected to high pressure homogenization. The organic solvent can then be removed by evaporation. The dispersion obtained can be further lyophilized. Suitable organic solvent include, for example, ketones, esters, ethers, chlorinated solvents, and other solvents known in the art. For example, the organic solvent can be methylene chloride or chloroform/ethanol (for example with a ratio of 1:9, 1:8, 1:7, 1:6, 1:5, 1:4, 1:3, 1:2, 1:1, 2:1, 3:1, 4:1, 5:1, 6:1, 7:1, 8:1, or 9:1).

### Other Components in the Nanoparticle Compositions

The nanoparticle compositions described herein can be present in a nanoparticle composition that includes other agents, excipients, or stabilizers. For example, to increase stability by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearyolphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

In some embodiments, the nanoparticle composition is suitable for administration to a human. In some embodiments, the nanoparticle composition is suitable for administration to a mammal such as, in the veterinary context, domestic pets and agricultural animals. There are a wide variety of suitable formulations of the nanoparticle composition (see. e.g., U.S. Pat. Nos. 5,916,596 and 6,096,331). The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the nanoparticle composition is formulated to have a pH range of about 4.5 to about 9.0, including for example pH ranges of any of about 5.0 to about 8.0, about 6.5 to about 7.5, and about 6.5 to about 7.0. In some embodiments, the pH of the nanoparticle composition is formulated to no less than about 6, including for example no less than about any of 6.5, 7, or 8 (such as about 8). The nanoparticle composition can also be made to be isotonic with blood by the addition of a suitable tonicity modifier, such as glycerol.

Dosing and Method of Administering the Allosteric mTOR Inhibitor

The dose of the allosteric mTOR inhibitor (such as a limus nanoparticle composition) administered to an individual (such as a human) may vary with the particular allosteric mTOR inhibitor, the mode of administration, the disease being treated, and the severity of symptoms. In some embodiments, the amount of the allosteric mTOR inhibitor is effective to reduce one or more symptoms exhibited by individuals having a disease, such as a mitochondrial-associated disorder. In some embodiments, the amount of the allosteric mTOR inhibitor is effective to prevent one or more symptoms associated with having a disease, such as a mitochondrial-associated disorder. In some embodiments, the amount of the allosteric mTOR inhibitor is effective to treat one or more symptoms associated with having a disease, such as a mitochondrial-associated disorder. In some embodiments, the amount of the allosteric mTOR inhibitor is effective to ameliorate one or more symptoms associated with having a disease, such as a mitochondrial-associated disorder. In some embodiments, the amount of the allosteric mTOR inhibitor is effective to alleviate one or more symptoms associated with having a disease, such as a mitochondrial-associated disorder. In some embodiments, the amount of the allosteric mTOR inhibitor is effective to delay onset of one or more symptoms associated with having a disease, such as a mitochondrial-associated disorder.

Responses of an individual and clinical benefit of the treatment methods described herein can be determined, for example, based on audiogram, magnetic resonance imaging, computed tomography, magnetic resonance spectroscopy, electroencephalography, electrocardiography, echocardiography, electroretinography, immunohistochemistry, and assessment of a biomarker.

In some embodiments, the amount of the allosteric mTOR inhibitor is sufficient to prolong survival of the individual. In some embodiments, the amount of the allosteric mTOR inhibitor (for example when administered alone) is sufficient to produce clinical benefit of more than about any of 50%, 60%, 70%, or 77% among a population of individuals treated with the allosteric mTOR inhibitor (such as a limus nanoparticle composition).

In some embodiments, the amount of the allosteric mTOR inhibitor (such as a limus nanoparticle composition) is below the level that induces a toxicological effect (i.e., an effect above a clinically acceptable level of toxicity) or is at a level where a potential side effect can be controlled or tolerated when the allosteric mTOR inhibitor is administered to the individual.

In some embodiments, the amount of the allosteric mTOR inhibitor (such as in the nanoparticle composition) is close to a maximum tolerated dose (MTD) of the allosteric mTOR inhibitor following the same dosing regimen. In some embodiments, the amount of the allosteric mTOR inhibitor is more than about any of 80%, 90%, 95%, or 98% of the MTD.

In some embodiments, the effective amount of allosteric mTOR inhibitor (such as in the nanoparticle composition) includes, but is not limited to, at least about any of 0.1 mg/m², 0.5 mg/m², 1 mg/m², 2 mg/m², 3 mg/m², 4 mg/m², 5 mg/m², 10 mg/m², 15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m², 125 mg/m², 130 mg/m², 135 mg/m², 140 mg/m², 145 mg/m², or 150 mg/m² of the allosteric mTOR inhibitor. In various embodiments, the allosteric mTOR inhibitor (such as in the nanoparticle composition) includes less than about any of 150 mg/m², 125 mg/m², 100 mg/m², 75 mg/m², 50 mg/m², 25 mg/m², 20 mg/m², 15 mg/m², 10 mg/m², 5 mg/m², 4 mg/m², 3 mg/m², or 2 mg/m² of the allosteric mTOR inhibitor (e.g., sirolimus). In some embodiments, the amount of allosteric mTOR inhibitor (such as in the nanoparticle composition) per administration is less than about any of 25 mg/m², 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², or 2 mg/m². In some embodiments, the effective amount of allosteric mTOR inhibitor (such as in the nanoparticle composition) is included in any of the following ranges: about 1 to about 5 mg/m², about 5 to about 10 mg/m², about 10 to about 25 mg/m², about 25 to about 50 mg/m², about 50 to about 75 mg/m², or about 75 to about 100 mg/m² (such as about any of 10 mg/m², 20 mg/m², or 30 mg/m²). In some embodiments, the effective amount of allosteric mTOR inhibitor (such as in the nanoparticle composition) is about 5 to about 200 mg/m², such as about 25 to about 75 mg/m², about 80 mg/m², about 85 mg/m², about 90 mg/m², about 95 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², or about 200 mg/m² of the allosteric mTOR inhibitor.

In some embodiments of any of the above aspects, the effective amount of allosteric mTOR inhibitor (such as in the nanoparticle composition) includes at least about any of 1 mg/kg, 2.5 mg/kg, 3.5 mg/kg, 5 mg/kg, 6.5 mg/kg, 7.5 mg/kg, 10 mg/kg, 15 mg/kg, 20 mg/kg. 25 mg/kg, 30 mg/kg, 35 mg/kg, 40 mg/kg, 45 mg/kg, 50 mg/kg, 55 mg/kg, or 60 mg/kg of the allosteric mTOR inhibitor. In various embodiments, the effective amount of allosteric mTOR inhibitor (such as in the nanoparticle composition) includes less than about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg of the allosteric mTOR inhibitor (e.g., sirolimus).

In some embodiments, the dosing frequencies for the administration of the nanoparticle compositions include, but are not limited to, daily, every two days, every three days, every four days, every five days, every six days, weekly without break, three out of four weeks, once every four weeks, once every three weeks, once every two weeks, or two out of three weeks. In some embodiments, the allosteric mTOR inhibitor is administered about once every 2 weeks, once every 3 weeks, once every 4 weeks, once every 6 weeks, or once every 8 weeks. In some embodiments, the allosteric mTOR inhibitor is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1, 8, and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 8 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on days 1 and 15 of a 28-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), on day 1 of a 21-day cycle. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), once per week. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), once every two weeks. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), once every three weeks. In some embodiments, the allosteric mTOR inhibitor (such as a nanoparticle limus drug) is administered at a dosage of about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), once every four weeks.

In some embodiments, the dosing frequency is once every two days for one time, two times, three times, four times, five times, six times, seven times, eight times, nine times, ten times, and eleven times. In some embodiments, the dosing frequency is once every two days for five times. In some embodiments, the allosteric mTOR inhibitor (such as in the nanoparticle composition) is administered over a period of at least ten days, wherein the interval between each administration is no more than about two days, and wherein the dose of the allosteric mTOR inhibitor (such as in the nanoparticle composition) at each administration is about 0.1 mg/m² to about 150 mg/m², including, for example, about 0.5 mg/m² to about 100 mg/m², about 5 mg/m² to about 100 mg/m², or about 1 mg/m² to about 30 mg/m² (such as about any of 10 mg/m², 20 mg/m², or 30 mg/m²), of the allosteric mTOR inhibitor.

The administration of the allosteric mTOR inhibitor (such as in the nanoparticle composition) can be extended over an extended period of time, such as from about a month up to about seven years. In some embodiments, the allosteric mTOR inhibitor (such as in the nanoparticle composition) is administered over a period of at least about any of 2, 3, 4, 5, 6, 7, 8, 9, 10, 11, 12, 18, 24, 30, 36, 48, 60, 72, or 84 months.

In some embodiments, the dosage of the allosteric mTOR inhibitor (e.g., sirolimus) in a nanoparticle composition can be in the range of 1-100 mg/m² when given on a 3 week schedule, or 1-100 mg/m² (such as 5-100 mg/m²) when given on a weekly schedule.

In some embodiments, the exemplary dosing schedules for the administration of the allosteric mTOR inhibitor (such as in the nanoparticle composition) include, but are not limited to, 150 mg/m², weekly, without break; 150 mg/m², weekly, 2 out of 3 weeks; 150 mg/m², weekly, 3 out of 4 weeks; 100 mg/m², weekly, without break; 100 mg/m², weekly, 2 out of 3 weeks; 100 mg/m², weekly, 3 out of 4 weeks; 75 mg/m², weekly, without break; 75 mg/m², weekly, 2 out of 3 weeks; 75 mg/m², weekly, 3 out of 4 weeks; 50 mg/m², weekly, without break; 50 mg/m², weekly, 2 out of 3 weeks; 50 mg/m², weekly, 3 out of 4 weeks. The dosing frequency of allosteric mTOR inhibitor may be adjusted over the course of the treatment based on the judgment of the administering physician.

In some embodiments, the individual is treated for at least about any of one, two, three, four, five, six, seven, eight, nine, or ten treatment cycles.

The allosteric mTOR inhibitors (such as in the nanoparticle compositions) described herein allow infusion of mTOR inhibitor to an individual over an infusion time that is shorter than about 24 hours. For example, in some embodiments, the mTOR inhibitor (such as in the nanoparticle composition) is administered over an infusion period of less than about any of 24 hours, 12 hours, 8 hours, 5 hours, 3 hours, 2 hours, 1 hour, 30 minutes, 20 minutes, or 10 minutes. In some embodiments, the allosteric mTOR inhibitor (such as in the nanoparticle composition) is administered over an infusion period of about 30 minutes.

The allosteric mTOR inhibitor (such as in the nanoparticle composition) can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the allosteric mTOR inhibitor may be used. In some embodiments, the allosteric mTOR inhibitor is administered intravenously. In some embodiments, the allosteric mTOR inhibitor is administered intraportally. In some embodiments, the allosteric mTOR inhibitor is administered intraarterially. In some embodiments, the allosteric mTOR inhibitor is administered intraperitoneally. In some embodiments, the allosteric mTOR inhibitor is administered intrahepatically. In some embodiments, the allosteric mTOR inhibitor is administered by hepatic arterial infusion. In some embodiments, the allosteric mTOR inhibitor is administered intravesicularly. In some embodiments, the allosteric mTOR inhibitor is administered subcutaneously. In some embodiments, the allosteric mTOR inhibitor is administered intrathecally. In some embodiments, the allosteric mTOR inhibitor is administered intrapulmonarily. In some embodiments, the allosteric mTOR inhibitor is administered intramuscularly. In some embodiments, the allosteric mTOR inhibitor is administered intratracheally. In some embodiments, the allosteric mTOR inhibitor is administered intraocularly. In some embodiments, the allosteric mTOR inhibitor is administered transdermally. In some embodiments, the allosteric mTOR inhibitor is administered intradermally. In some embodiments, the allosteric mTOR inhibitor is administered orally. In some embodiments, the allosteric mTOR inhibitor is administered by inhalation.

In some embodiments when the limus nanoparticle composition is administered intravesicularly, the dosage of an allosteric mTOR inhibitor (such as a limus drug, e.g., sirolimus) in a nanoparticle composition can be in the range of about 30 mg to about 400 mg in volume of about 20 to about 150 ml, for example retained in the bladder for about 30 minutes to about 4 hours. In some embodiments, the nanoparticle composition is retained in the bladder for about 30 minutes to about 4 hours, including for example about 30 minutes to about 1 hour, about 1 hour to about 2 hours, about 2 hours to about 3 hours, or about 3 hours to about 4 hours.

In some embodiments, the dosage of allosteric mTOR inhibitor (such as in the nanoparticle composition) is about 100 to about 400 mg, for example about 100 mg, about 200 mg, about 300 mg, or about 400 mg. In some embodiments, the limus drug (such as in the limus nanoparticle composition) is administered at about 100 mg weekly, about 200 mg weekly, about 300 mg weekly, about 100 mg twice weekly, or about 200 mg twice weekly. In some embodiments, the administration is further followed by a monthly maintenance dose (which can be the same or different from the weekly doses).

In some embodiments when the limus nanoparticle composition is administered intravenously, the dosage of the allosteric mTOR inhibitor (such as a limus drug, e.g., sirolimus) in a nanoparticle composition can be in the range of about 30 mg to about 400 mg. The allosteric mTOR inhibitors described herein allow infusion of the allosteric mTOR inhibitors to an individual over an infusion time that is shorter than about 24 hours. For example, in some embodiments, the allosteric mTOR inhibitor (such as in the nanoparticle composition) is administered over an infusion period of less than about any of 24 hours, 12 hours, 8 hours, 5 hours, 3 hours, 2 hours, 1 hour, 30 minutes, 20 minutes, or 10 minutes. In some embodiments, the allosteric mTOR inhibitor (such as in the nanoparticle composition) is administered over an infusion period of about 30 minutes to about 40 minutes.

### Kits, Medicines, Compositions, and Unit Dosages

The invention also provides kits, medicines, compositions, and unit dosage forms for use in any of the methods described herein.

Kits of the invention include one or more containers comprising an allosteric mTOR inhibitor (or unit dosage forms and/or articles of manufacture), further comprise instructions for use in accordance with any of the methods described herein. The kit may further comprise a description of selection an individual suitable or treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises a) an allosteric mTOR inhibitor (such as a limus drug), and b) instructions for administering the allosteric mTOR inhibitor for treatment of a disease, such as a mitochondrial-associated disorder. In some embodiments, the kit comprises a) an allosteric mTOR inhibitor (such as a limus drug), b) another therapeutic agent, and c) instructions for administering (such as administering subcutaneously or intravenously) the allosteric mTOR inhibitor and the other agents for treatment of a disease, such as a mitochondrial-associated disorder. The allosteric mTOR inhibitor and the other agents can be present in separate containers or in a single container. For example, the kit may comprise one distinct composition or two or more compositions wherein one composition comprises nanoparticles and one composition comprises another agent.

For example, in some embodiments, the kit comprises a) a composition comprising nanoparticles comprising an allosteric mTOR inhibitor (such as a limus drug) and an albumin (such as human serum albumin), and b) instructions for administering the nanoparticle composition for treatment of a disease, such as a mitochondrial-associated disorder. In some embodiments, the kit comprises a) a composition comprising nanoparticles comprising an allosteric mTOR inhibitor (such as a limus drug) and an albumin (such as human serum albumin), b) another therapeutic agent, c) and instructions for administering (such as administering subcutaneously or intravenously) the nanoparticle composition and the other agents for treatment of a disease, such as a mitochondrial-associated disorder. The nanoparticles and the other agents can be present in separate containers or in a single container. For example, the kit may comprise one distinct composition or two or more compositions wherein one composition comprises nanoparticles and one composition comprises another agent.

The kits of the invention are in suitable packaging. Suitable packaging include, but is not limited to, vials, bottles, jars, flexible packaging (e.g., seled Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the allosteric mTOR inhibitor (such as a nanoparticle composition) generally include information as to dosage, dosing schedule, and route of administration for the intended treatment. The containers may be unit doses, bulk packages (e.g., multi-dose packages) or sub-unit doses. For example, kits may be provided that contain sufficient dosages of the allosteric mTOR inhibitor (such as a limus drug, e.g., sirolimus) as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the allosteric mTOR inhibitor (such as a limus drug) and pharmaceutical compositions and instructions for use and packaged in quantities sufficient for storage and use in pharmacies, for example, hospital pharmacies and compounding pharmacies.

Also provided are medicines, compositions, and unit dosage forms useful for the methods described herein. In some embodiments, there is provided a medicine (or composition) for use in treating an individual having a disease, such as a mitochondrial-associated disorder, comprising allosteric mTOR inhibitor (such as a nanoparticle composition).

### EXEMPLARY EMBODIMENTS

### Embodiment 1

A method of treating an individual having a mitochondrial-associated disorder comprising administering to the individual an effective amount of an allosteric mTOR inhibitor.

### Embodiment 2

The method of embodiment 1, wherein the individual having a mitochondrial-associated disorder has one or more of the following: an ataxia, a kidney disorder, a liver disorder, a metabolic disorder, a myopathy, a neuropathy, a myelopathy, an encephalopathy, or an oxidative phosphorylation disorder.

### Embodiment 3

The method of embodiment 1 or 2, wherein the individual having a mitochondrial-associated disorder has Leigh syndrome.

### Embodiment 4

The method of embodiment 3, wherein Leigh syndrome is maternally inherited Leigh syndrome.

### Embodiment 5

The method of embodiment 3 or 4, wherein Leigh syndrome is infantile onset Leigh syndrome, juvenile onset Leigh syndrome, or adult onset Leigh syndrome.

### Embodiment 6

The method of embodiment 1 or 2, wherein the individual having a mitochondrial-associated disorder has MELAS syndrome.

### Embodiment 7

The method of embodiment 1 or 2, wherein the individual having a mitochondrial-associated disorder has NARP syndrome.

### Embodiment 8

The method of embodiment 1, wherein the individual having a mitochondrial-associated disorder has one or more of the following: an aging disorder, an autism spectrum disorder, a chronic inflammatory disorder, diabetes mellitus, or a fatty acid oxidation disorder.

### Embodiment 9

The method of any one of embodiments 1-8, wherein the individual having a mitochondrial-associated disorder has a mitochondrial DNA mutation-associated disorder.

### Embodiment 10

The method of any one of embodiments 1-9, wherein the individual having a mitochondrial-associated disorder has a nuclear DNA mutation-associated disorder.

### Embodiment 11

The method of any one of embodiments 1-10, wherein the individual having a mitochondrial-associated disorder has an X chromosome mutation-associated disorder.

### Embodiment 12

The method of any one of embodiments 1-11, wherein the individual is about one month old to about thirty years old.

### Embodiment 13

The method of any one of embodiments 1-12, wherein the age of onset of one or more mitochondrial-associated disorder symptoms in the individual is between about three months old and about two years old.

### Embodiment 14

The method of any one of embodiments 1-13, wherein the individual is a male.

### Embodiment 15

The method of any one of embodiments 1-14, wherein the individual has a mutation in one or more of the following genes: LRPPRC, MT-ATP6, MT-ND1, MT-ND2, MT-ND3, MT-ND5, MT-ND6, MT-TL1, MT-TH, MT-TV, NDUFS1, NDUFS2, NDUFS3, NDUFS4, NDUFS7, NDUFS8, or SURF1.

### Embodiment 16

The method of any one of embodiments 1-15, wherein the individual is selected for treatment based on the ratio of lactate to pyruvate in their blood, plasma, cerebrospinal fluid, or urine.

### Embodiment 17

The method of embodiment 16, wherein the ratio of lactate to pyruvate is at least 10:1.

### Embodiment 18

The method of embodiment 16 or 17, wherein the ratio of lactate to pyruvate is at least 20:1.

### Embodiment 19

A method of inhibiting cellular glucose consumption in an individual comprising administering to the individual an effective amount of an allosteric mTOR inhibitor.

### Embodiment 20

A method of treating an individual having a metabolic disorder comprising administering to the individual an effective amount of an allosteric mTOR inhibitor.

### Embodiment 21

A method of treating an individual having a disease comprising administering to the individual an effective amount of an allosteric mTOR inhibitor, wherein the disease is selected from the group consisting of fetal dilated cardiomyopathy, tuberous sclerosis complex (TSC) and related disorders, childhood onset cardiomyopathy. Noonan syndrome, polycystic kidney disease, age-related and genetically induced hypertrophic cardiomyopathy, and a rheumatic disease.

### Embodiment 22

The method of any one of embodiments 1-21, wherein the allosteric mTOR inhibitor is in a composition comprising nanoparticles comprising the allosteric mTOR inhibitor and an albumin.

### Embodiment 23

The method of any one of embodiments 1-22, wherein the allosteric mTOR inhibitor is a limus drug.

### Embodiment 24

The method of embodiment 23, wherein the limus drug is sirolimus.

### Embodiment 25

The method of any one of embodiments 1-24, wherein the effective amount of allosteric mTOR inhibitor is about 1 mg/m² to about 150 mg/m².

### Embodiment 26

The method of any one of embodiments 1-25, wherein the effective amount of allosteric mTOR inhibitor is administered weekly.

### Embodiment 27

The method of any one of embodiments 1-25, wherein the effective amount of allosteric mTOR inhibitor is administered once every two weeks.

### Embodiment 28

The method of any one of embodiments 1-25, wherein the effective amount of allosteric mTOR inhibitor is administered daily.

### Embodiment 29

The method of any one of embodiments 1-25, wherein the effective amount of allosteric mTOR inhibitor is administered once every three days.

### Embodiment 30

The method of any one of embodiments 1-29, wherein the effective amount of allosteric mTOR inhibitor is administered intravenously, intraarterially, intraperitoneally, intravesicularly, subcutaneously, intrathecally, intrapulmonarily, intramuscularly, intratracheally, intraocularly, transdermally, intradermally, orally, intraportally, intrahepatically, by hepatic arterial infusion, or by inhalation.

### Embodiment 31

The method of embodiment 30, wherein the effective amount of allosteric mTOR inhibitor is administered intravenously.

### Embodiment 32

The method of any one of embodiments 22-31, wherein the nanoparticles in the composition have an average diameter of no greater than about 150 nm.

### Embodiment 33

The method of embodiment 32, wherein the nanoparticles in the composition have an average diameter of no greater than about 120 nm.

### Embodiment 34

The method of any one of embodiments 22-33, wherein the allosteric mTOR inhibitor in the nanoparticles is associated with the albumin.

### Embodiment 35

The method of any one of embodiments 22-34, wherein the weight ratio of albumin and allosteric mTOR inhibitor in the nanoparticle composition is about 1:1 to about 9:1.

### Embodiment 36

The method of embodiment 35, wherein the weight ratio of albumin and allosteric mTOR inhibitor in the nanoparticle composition is about 8:1, about 8.5:1, or about 9:1.

### Embodiment 37

The method of any one of embodiments 22-36, wherein the albumin is human albumin.

### Embodiment 38

The method of any one of embodiments 22-36, wherein the albumin is human serum albumin.

### Embodiment 39

The method of any one of embodiments 1-38, wherein the individual is human.

### Embodiment 40

The method of any one of embodiments 1-39, wherein the individual has not been previously treated with an allosteric mTOR inhibitor.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

### Example 1

### Study of the Effect of Nab-Sirolimus on S6 Phosphorylation

This study is designed to assess the effect of nab-sirolimus on S6 phosphorylation. Human mitochondrial disease line fibroblasts are treated with nab-sirolimus at varying doses for 48 hours. The level of ribosomal protein S6 (S6) phosphorylation (pS6) is compared among the varying treatment doses. Phosphorylation of S6 (measured as a ratio of the intensity of pS6 to total S6) is a well-established readout of mTOR activity in both normal cells and in the setting of mitochondrial disease (see Johnson et al., Science, 342, 2013). It is previously established that 100 ng/mL rapamycin robustly inhibits S6 phosphorylation (Johnson et al., Science, 342, 2013). Inhibition of S6 phosphorylation by nab-sirolimus is to be tested and compared to rapamycin and other allosteric mTOR inhibitors such as torin 1. Comparative dose responses for membrane potential and morphology will also be performed.

### Example 2

### Study of Nab-Sirolimus in the NDUSF4 Knockout Mouse Model of Leigh Syndrome

This study is designed to assess the efficacy and safety of nab-sirolimus (also referred to as ABI-009) in the Ndufs4 knockout (Ndufs4 KO) mouse model of Leigh syndrome. Ndufs4 KO mice appear to develop normally until around five weeks of age when they develop symptoms of ataxia. Death results by approximately seven weeks of age. The mice have a diminished capacity for oxidative phosphorylation, and exhibit many symptoms of Leigh syndrome in humans, including a retarded growth rate, lethargy, loss of motor skill, blindness, and elevated serum lactate.

Ndufs4 KO mice are administered varying doses of nab-sirolimus. Clinical benefit, as compared to control Ndufs4 KO mice, is measured.

### Example 3

### Study of Nab-Sirolimus in the POLG Knockout Mouse Model of MELAS Syndrome

This study is designed to assess the efficacy and safety of nab-sirolimus (also referred to as ABI-009) in the polymerase gamma (POLG) knockout (POLG KO) mouse model of MELAS syndrome. Polymerase gamma is a mitochondrial DNA polymerase and is a causal disease gene in MELAS and other human mitochondrial diseases.

POLG KO mice are administered varying doses of nab-sirolimus. Clinical benefit, as compared to control POLG KO mice, is measured.

### Example 4A

Phase I Open-Label, Multi-Center Study of the Safety and Clinical Activity of Single and Multiple Doses of Nab-Sirolimus for Treating Patients with Leigh Syndrome

This example describes a phase I open-label, multi-center study of the safety and clinical activity of single and multiple doses of nab-sirolimus for treating patients with Leigh syndrome.

The study is designed as a two-part study of the safety and efficacy of a single dose and multiple doses of intravenously administered nab-sirolimus (also known as ABI-009 or nab-rapamycin). In part 1, enrolled patients are split into three groups and each patient receives a single dose of nab-sirolimus (dose for group A: 10 mg/m², dose for group B: 20 mg/m², and dose for group C: 30 mg/m²). Patients are monitored for safety for a minimum of 7 days following administration of nab-sirolimus. Patients who experience unacceptable toxicity during part 1 will not continue to the multi-dose phase (part 2). In part 2, patients are treated with the maximum tolerated dose (MTD) identified in part 1 of the study. Patients receive nab-sirolimus once per week over a 30-minute infusion for up to 6 months and will be followed for safety and clinical activity. Dose reduction is permitted if unacceptable toxicity is observed. Patients who show evidence of clinical activity and acceptable toxicity are permitted to continue to receive nab-sirolimus in an extension protocol. In part 2, patients receive nab-sirolimus at the discretion of the investigator, until unacceptable toxicity, disease progression, until in the opinion of the investigator the patient is no longer benefiting from therapy, at the Sponsor's request, withdrawal of consent, or death.

An initial laboratory evaluation is performed for each patient. The initial laboratory evaluation includes assessment of: (1) blood glucose; (2) blood electrolytes; (3) blood counts; (4) blood lactate; (5) blood ammonia; (6) blood and urine metabolic screens; and (7) blood and urine ketones.

An additional secondary laboratory evaluation is performed for each patient. The secondary laboratory evaluation includes assessment of: (1) blood and cerebral spinal fluid (CSF) lactate; (2) blood pyruvate; (3) blood lactate to pyruvate ratio; (4) blood, urine, and CSF amino acids; (5) urine and CSF organic acids; (6) blood and urine carnitine; (7) blood and urine ketones; (8) blood free fatty acids; (9) mitochondrial DNA point mutations; and (10) genetic point mutations.

Patient eligibility is based on meeting all of the following: (1) diagnosis of a mitochondrial-associated disorder, such as Leigh syndrome (including, for example, MRI and/or genetically confirmed Leigh syndrome); (2) MRI confirmation of necrotizing encephalopathy; (3) moderately severe disease based on NPMDS score of >15 on Sections I through III, inclusive; (4) male or female patients; (5) ≥1 and ≤17 years of age at the time of enrollment: (6) body weight ≥5 kg (11 lbs); life expectancy of at least 6 months, as determined by the Investigator; (7) laboratory values obtained at the screening evaluation as follows: (a) absolute neutrophil count >1.5×109 cells/L, (b) serum creatinine <1.5 mg/dL (<132.6 μmol/L) or Cockcroft-Gault glomerular filtration rate (GFR) >60 mL/min, (c) liver function tests: AST and/or ALT <1.5× the upper limit of normal (ULN), and/or total bilirubin <than the ULN, (d) fasting serum triglycerides <300 mg/dL (<3.39 mmol/L), (c) fasting serum cholesterol <350 mg/dL (<9.07 mmol/L): (8) if receiving prescribed medications to prevent or treat seizures, the patient must be receiving stable doses for at least 30 days prior to the screening visit; (9) non-pregnant and non-breast feeding women of child-bearing potential (WOCBP); (10) must agree to use effective contraception without interruption from 28 days prior to starting study drug and throughout the treatment period and for 6 months following the last dose of study drug; (11) must agree to use a second form of birth control, even if she has had a tubal ligation; (12) must have a negative urine or serum pregnancy test (β-hCG) result at screening; (13) must agree to ongoing pregnancy testing during the course of the study and after the end of study treatment; (14) males must practice abstinence or agree to use a condom (with a spermicide) during sexual contact with any pregnant female or any woman of childbearing potential (WOCBP) while participating in the study and for 6 months following the last dose of study drug. A second form of birth control is required, even if the male patient has undergone a successful vasectomy; (15) completed informed consent process, including signing the Institutional Review Board (IRB)/Ethics Committee (EC)-approved informed consent document and Assent Form, if applicable; and (16) for patients under the age of consent, a parent or guardian of the patient who is able to comply with clinical trial instructions and requirements, and who will commit to all of the follow-up visits for the duration of the study.

Patient exclusion is based on meeting any of the following: (1) confirmed or suspected diagnosis of inborn error of metabolism; (2) previous tracheostomy, ventilator-dependent, or use of noninvasive ventilator support within one month prior to enrollment; (3) renal insufficiency that, in the opinion of the Investigator, requires or may require dialysis during the treatment and follow-up periods (patients who develop renal insufficiency during the course of treatment will be discontinued from study drug); (4) severe end-organ hypo-perfusion syndrome (secondary to cardiac failure) resulting in lactic acidosis; (5) prior exposure to nab-sirolimus, sirolimus, everolimus, or any other known rapamycin derivative/rapalog, or previous treatment with any known mTOR inhibitor; (6) patients who are breast feeding or have a confirmed or suspected pregnancy; (7) treatment with any investigational drug (i.e., a drug for which there is no approved indication) within 30 days prior to receiving the first dose of study drug; (8) current use of supplements, including super-fortified foods and/or beverages that include coenzyme Q10. Vitamin C, Vitamin E, and/or idebanone (all such supplements must be discontinued prior to enrollment); (9) known hypersensitivity to nab-sirolimus, any of its excipients, or any rapamycin derivative; (10) patients with confirmed or suspected intracranial pressure, pseudotumor cerebri (PTC), and/or papilledema; (11) clinically significant ECG findings at the time of screening; (12) any uncontrolled serious illness or psychiatric condition, medical condition, or other medical history, including abnormal laboratory test results which, in the opinion of the Investigator, would be likely to interfere with the patient's participation in the study, or with the interpretation of the results of the study: (13) currently active malignancy (other than adequately treated non-melanoma skin cancers, i.e., squamous cell and/or basal cell carcinoma, carcinoma in situ of the cervix, or other adequately treated carcinoma in situ) and/or ongoing treatment for malignancy are ineligible (patients are not considered to have a currently active malignancy if they have completed therapy and are free of disease for ≥1 year); (14) recent infection requiring systemic anti-infective treatment that was completed ≤14 days prior to enrollment (with the exception of uncomplicated urinary tract infection or upper respiratory tract infection); (15) uncontrolled diabetes mellitus, as defined by HbA1c >8% despite adequate therapy; (16) myocardial infarction during the 6 months prior to enrollment; (17) symptomatic congestive heart failure (CHF), unstable angina pectoris, cardiac arrhythmia, uncontrolled hypertension, or unstable coronary artery disease; (18) history of interstitial lung disease and/or pneumonitis, or pulmonary hypertension; (19) use of strong inhibitors and/or inducers of cytochrome P450 (CYP) 3A4 (CYP3A4) and/or p-glycoprotein (p-GP) within the 14 days prior to receiving the first dose of nab-sirolimus (additionally, use of any known CYP3A4 substrates with a narrow therapeutic window, such as fentanyl, alfentanil, astemizole, cisapride, dihydroergotamine, pimozide, quinidine, terfenadine, within the 14 days prior to receiving the first dose of study drug); (20) planned vaccination with live vaccines during treatment with nab-sirolimus, and for 4 weeks after receipt of the last dose of nab-sirolimus (live vaccines may include, but are not limited to, measles, mumps, rubella, oral polio, BCG (Bacillus Calmette-Guerin), yellow fever, varicella, and T21a typhoid); (21) known human immunodeficiency virus (HIV), active hepatitis B or hepatitis C infection(s); (22) active participation in an investigational drug trial for mitochondrial disease within 30 days prior to enrollment (or within 90 days for a trial with an investigational biologic), or disease-related surgical intervention within 30 days prior to enrollment; or (23) any condition (e.g., known or suspected poor compliance, psychological instability, and geographical location) that, in the opinion of the Investigator, may affect the patient's ability to fully comply with all requirements of the study.

The End of Study (EOS) is defined as (1) either the date of the last visit of the last patient to complete the study or (2) the date of collection of the last data point from the last patient that is required for primary, secondary, and/or exploratory analyses, as pre-specified in the protocol.

End of Treatment (EOT) for a patient is defined as the date of the last dose of nab-sirolimus. The EOT for a patient is when safety assessments and procedures are performed after the last treatment, which must occur at least 4 weeks (±7 days) after the last dose of nab-sirolimus.

Follow-up period is the on-study time period after the EOT Visit. All patients that discontinue study drug and have not withdrawn full consent to participate in the study will continue in the follow-up phase for collection of survival data and clinical activity of nab-sirolimus. Follow up will continue approximately every 12 weeks (+/−3 weeks), until death, withdrawal of consent, or the study closes, whichever is the earliest. This evaluation may be made by record review and/or telephone contact.

In part 2 of the study, up to 2 dose reductions will be permitted. For example, if a patient is administered 30 mg/m², the two-step dose reduction is an initial dose reduction to 20 mg/m² and then, if unacceptable toxicity persists, a second dose reduction to 10 mg/m². If a patient is administered, for example, 20 mg/m², the two-step dose reduction is an initial dose reduction to 10 mg/m² and then, if unacceptable toxicity persists, a second dose reduction to 5 mg/m². If a patient is administered, for example, 10 mg/m², the two-step dose reduction is an initial dose reduction to 5 mg/m² and then, if unacceptable toxicity persists, a second dose reduction to 2.5 mg/m².

Following the initial dose reduction, if toxicity improves within 2 weeks, the patient will continue to receive the initial reduced dose unless further toxicity develops. If toxicity does not improve within 2 weeks of the first dose reduction, the patient will be administered, if at all, a dose based on a second dose reduction. Following the second dose reduction, if toxicity does improve within 1 week or resolve to an acceptable level (as determined by the investigator), administration of nab-sirolimus will be terminated. If, following dose reduction, toxicity has improved to an acceptable level (as determined and documented by the Investigator), patients will continue to receive therapy until disease progression, new or recurrent unacceptable toxicity, until in the opinion of the Investigator the patient is no longer benefiting from therapy, at the Sponsor's request, or at the discretion of the patient.

Primary endpoints for part 1 include safety and tolerability, as well as determination of the maximum tolerated (single) dose (MTD). Safety evaluations include a determination of, for example, serious adverse events (SAEs)/adverse events (AEs), laboratory parameter assessments, physical examinations, vital signs, and ECGs. Primary endpoints for part 2 include safety and tolerability.

Secondary endpoints for the study include, but are not limited to: clinical activity, as determined by examining the change from baseline in the Newcastle Pediatric Mitochondrial Disease Scale (NPMDS), Gross Motor Function Measure (GMFM), and Quality of Life (PedsQL) at 6 months; change in neuromuscular function, as determined by the Barry-Albright Dystonia Scale; change in respiratory function, as determined by oxygen (O2) saturation and need for tracheostomy; change in cardiac function: disease progressions as determined by brain MRI; morbidity (overall patient survival); mortality (number of hospitalizations); and pharmacokinetic/pharmacodynamic relationships for the primary clinical activity endpoints, as well as for select secondary and/or exploratory and safety endpoints, may also be examined.

Exploratory objectives for the study include, but are not limited to, alterations of biomarkers in blood (e.g., plasma) and other patient samples (e.g., CSF), such as lactate; ketones (e.g., acetoacetate and β-hydroxybutyrate); metabolites of the glycolytic pathway (e.g., phosphofructokinase, hexokinase, pyruvate kinase, glucose transporters GLUT 1 to GLUT 5); lymphocytes; total, reduced, and oxidized glutathione peroxidase enzyme activities; leukocytes; pyruvate dehydrogenase enzyme activity; purified mitochondria isolated from small muscle biopsy; cytochrome C oxidase activity; and respiratory chain enzyme activity.

Adverse events will be graded by National Cancer Institute (NCI) Common Terminology Criteria for Adverse Events. Physical examination, vital signs, ECG, laboratory assessments (e.g., serum chemistry, hematology) will be monitored.

After disease progression has been demonstrated, patients will be followed for survival every 12 weeks, or more frequently as needed, until death, withdrawal of consent, or the study closes, whichever is earliest.

Whole blood samples will be collected for determination of rapamycin concentration. Collected samples are stored frozen at temperatures between −20° C., and −80° C. until shipment for analysis at the central laboratory to be designated by the Sponsor. The whole blood samples are analyzed for total (free+bound) rapamycin using high-performance liquid chromatography-tandem mass spectrometry (HPLC-MS-MS).

In Part 1 of the study, samples will be collected immediately pre-dose on treatment day 1 (time 0: before infusion of nab-sirolimus), during infusion (times: 15 minutes and 30 minutes—just before the end of the infusion), and post-infusion (times: 1.0, 2, 4, 8, 24, 48, 72, 96, and 168 hours after completion of the infusion). In Part 2 of the study, samples will be collected immediately pre-dose on treatment day 1 and immediately pre-dose on any subsequent treatment administrations, during infusion (times: 15 minutes and 30 minutes—just before the end of the infusion), and post-infusion (times: 1.0, 1.5, 2, 4, 6, 8, 24, 48, 72, 96, and 168 hours after completion of the infusion). The concentration-versus-time data for rapamycin in whole blood will be analyzed using a noncompartmental analysis technique and WinNonlin software. Calculated parameters will include peak concentration (Cmax), half-life (t1/2), area under the concentration-time curve (AUC), clearance (CL), and steady-state volume of distribution (Vss). A simple regression model will be applied to assess the relationship of the pharmacokinetic parameters with dose. Pharmacokinetic/pharmacodynamics relationships for the primary clinical activity endpoints, as well as for select secondary and/or exploratory and safety endpoints, will also be evaluated.

Further, gene mutation status of the individuals will be assessed. The gene mutation status is identified via next-generation sequencing experiments from individuals in the clinical study. Additionally, correlative research is performed to assess the rate of a gene mutation status and assess the association between the gene mutation status and clinical outcome for individuals with the gene mutation status.

Prior to registration, individuals are assessed in a CLIA certified lab for gene mutations, such as, for example, at least one gene selected from: BCS1L, COX15, FOXRED1, GFM1, LRPPRC, MT-ATP6, MT-ND1, MT-ND2, MT-ND3, MT-ND5, MT-ND6, MT-TL1, MT-TH, MT-TV, NDUFS1, FA2, NDUFA9, NDUFA10, NDUFA12, NDUFAF2, NDUFAF6, NDUFS3, NDUFS4, NDUFS7, NDUFS8, NDUFV1, PDSS2, SDHA, and SURF1.

An archival paraffin embedded (PPFE) tissue sample may optionally be obtained from each individual.

Biomarkers (such as metabolite levels, enzyme and/or coenzyme activity levels) are evaluated for each individual on Day 1 of cycle 1, Day 1 (±3 days) of cycle 2, and Day 1 (±3 days) of Cycle 3 and then every 2 cycles afterward. A blood sample is collected from each individual to analyze circulating (e.g., cell-free) DNA before and after the entire course of treatment.

Various biological samples are collected from each individual during the course of the study (e.g., before treatment, on-treatment, and post-treatment), and the biological samples are used to assess the mutational status and level of relevant biomarkers. On-treatment biological samples may be collected from the individual, for example, on Day 1 of cycle 1, Day 1 (±3 days) of cycle 2, and Day 1 (±3 days) of Cycle 3 and then every 2 cycles afterward. A blood sample and or tissue sample is collected from each individual before and after the treatment. The DNA samples are analyzed using next-generation sequencing methods to assess the prevalence of gene mutations identified in the mitochondrial or nuclear DNA over time as a measure of response to the treatment. Additionally, fresh or archival (such as PPFE) muscle biopsy and/or skin biopsy samples are collected from each individual before the treatment, and optionally during the course of the treatment (i.e. on-treatment). The on-treatment muscle biopsy and/or skin biopsy samples are used to assess pharmacodynamics effects of nab-rapamycin in the individuals. Post-treatment muscle biopsy and/or skin biopsy samples are collected from each individual at the time of disease progression after response to the treatment to assess mechanisms of resistance, including secondary mutations, genomic amplifications, or gene deletion events.

Correlative research is performed to determine association of the treatment with clinical benefit and/or quality of life and an individual gene mutation status for the overall group of patients. Quality of life is assessed prior to review of treatment response and discussions of patient's general health since last treatment evaluation. Quality of life is measured using the EORTC QLQ-C30, a 30-item patient-report questionnaire about patient ability to function, symptoms related to the mitochondrial-associated disorder and its treatment, overall health and quality of life, and perceived financial impact of the syndrome and its treatment. Scale score trajectories of the quality of life over time are examined using stream plots and mean plots with standard deviation error bars. Changes from baseline at each cycle is statistically tested using paired t-tests, and standardized response means is interpreted after applying Middel's (2002) adjustment using Cohen's (1988) cutoffs: <0.20=trivial; 0.20-<0.50=small; 0.5-<0.8=moderate; and ≥0.8=large. Rate of individual mTOR-activating aberrations is described, and association with confirmed response is investigated using a Fisher's exact test. Associations with time to progression and overall survival are investigated using log-rank tests. One-sided p-values≤0.10 are considered statistically significant throughout.

### Example 4B

Phase I Open-Label, Multi-Center Study of the Safety and Clinical Activity of Nab-Sirolimus in Patients with Genetically Confirmed Leigh Syndrome

This example describes a phase I open-label, multi-center study of the safety and clinical activity of nab-sirolimus for treating patients with Leigh syndrome.

The study is designed as a dose-escalation study of the safety and efficacy of intravenously administered nab-sirolimus (also known as ABI-009 or nab-rapamycin). Enrolled patients are divided into three groups: Group 1 (n=4), 10 mg/m² nab-sirolimus administered IV once per week or once every 2 weeks for up to 26 weeks; Group 2 (n=4), 20 mg/m² nab-sirolimus administered IV once per week or once every 2 weeks for up to 26 weeks; and Group 3 (n=4), 30 mg/m² nab-sirolimus administered IV once per week or once every 2 weeks for up to 26 weeks. The ongoing safety of each dose level is assessed before patients are enrolled at the subsequent next higher dose level. A minimum of 2 patients in Group 1 are treated and followed for safety for at least 4 weeks before enrollment is initiated in Group 2. A minimum of 2 patients in Group 2 are treated and followed for safety for at least 4 weeks before enrollment is initiated in Group 3. At the discretion of the Sponsor, additional patients may be enrolled at any dose level to collect additional safety data or to further assess any toxicity that may occur. If unacceptable toxicity is observed at any dose level, dose reduction is permitted (per dose reduction guidelines for toxicity, provided below). Patients receive nab-sirolimus, at the discretion of the Investigator, until (1) unacceptable toxicity or disease progression, (2) in the opinion of the Investigator the patient is no longer benefiting from therapy. (3) at the Sponsor's request. (4) withdrawal of consent, or (5) death. After 6 months of treatment, evidence of disease progression may be added to the reasons for nab-sirolimus discontinuation. Patients who show evidence of clinical activity at the 26-week evaluation are permitted to continue to receive nab-sirolimus in an extension protocol.

Patient eligibility is based on meeting all of the following criteria: (1) diagnosis of Leigh syndrome, with genetic confirmation (with no variants of uncertain significance); (2) MRI confirmation of necrotizing encephalopathy; (3) moderately severe disease based on NPMDS score of >15 on Sections I through III, inclusive; (4) male or female patients; (5) ≥1 and ≤17 years of age at the time of enrollment; (6) body weight ≥5 kg (11 lbs); life expectancy of at least 12 months, as determined by the Investigator; (7) laboratory values obtained at the screening evaluation as follows: (a) absolute neutrophil count >1.5×10⁹ cells/L, (b) serum creatinine <1.5 mg/dL (<132.6 μmol/L) or Cockcroft-Gault glomerular filtration rate (GFR) >60 mL/min, (c) liver function tests: AST and/or ALT <1.5× the upper limit of normal (ULN), and/or total bilirubin <than the ULN. (d) fasting serum triglycerides <300 mg/dL (<3.39 mmol/L), (e) fasting serum cholesterol <350 mg/dL (<9.07 mmol/L); (8) if receiving prescribed medications to prevent or treat seizures, the patient must be receiving stable doses for at least 30 days prior to the screening visit; (9) non-pregnant and non-breast feeding women of child-bearing potential (WOCBP): (a) must agree to use effective contraception without interruption from 28 days prior to starting nab-sirolimus and throughout the treatment period and for 6 months following the last dose of nab-sirolimus; (b) must agree to use a second form of birth control, even if she has had a tubal ligation; (c) must have a negative urine or serum pregnancy test (β-hCG) result at screening; and (d) must agree to ongoing pregnancy testing during the course of the study and after the end of study treatment; (10) males must practice abstinence or agree to use a condom (with a spermicide) during sexual contact with any pregnant female or any woman of childbearing potential (WOCBP) while participating in the study and for 6 months following the last dose of nab-sirolimus, and a second form of birth control is required, even if the male patient has undergone a successful vasectomy; (11) completed informed consent process, including signing the Institutional Review Board (IRB)/Ethics Committee (EC)-approved informed consent document and Assent Form, if applicable; and (12) for patients under the age of consent, a parent or guardian of the patient who is able to comply with clinical trial instructions and requirements, and who will commit to all of the follow-up visits for the duration of the study.

Patient exclusion is based on meeting any of the following criteria: (1) confirmed or suspected diagnosis of inborn error of metabolism; (2) previous tracheostomy, ventilator-dependent, or use of noninvasive ventilator support within one month prior to enrollment; (3) renal insufficiency that, in the opinion of the Investigator, requires or may require dialysis during the treatment and follow-up periods; (4) severe end-organ hypo-perfusion syndrome (secondary to cardiac failure) resulting in lactic acidosis: (5) prior exposure to nab-sirolimus, sirolimus, everolimus, or any other known rapamycin derivative/rapalog, or previous treatment with any known mTOR inhibitor; (6) patients who are breast feeding or have a confirmed or suspected pregnancy; (7) treatment with any investigational drug (i.e., a drug for which there is no approved indication) within 30 days prior to receiving the first dose of nab-sirolimus; (8) current use of supplements, including super-fortified foods and/or beverages that include coenzyme Q10, Vitamin C, Vitamin E, and/or idebanone (all such supplements must be discontinued prior to enrollment); (9) known hypersensitivity to nab-sirolimus, any of its excipients, or any rapamycin derivative; (10) patients with confirmed or suspected intracranial pressure, pseudotumor cerebri (PTC)/idiopathic intracranial hypertension, and/or papilledema; (11) clinically significant ECG findings at the time of screening; (12) any uncontrolled serious illness or psychiatric condition, medical condition, or other medical history, including abnormal laboratory test results which, in the opinion of the Investigator, would be likely to interfere with the patient's participation in the study, or with the interpretation of the results of the study; (13) currently active malignancy (other than adequately treated non-melanoma skin cancers. i.e., squamous cell and/or basal cell carcinoma, carcinoma in situ of the cervix, or other adequately treated carcinoma in situ) and/or ongoing treatment for malignancy are ineligible (patients are not considered to have a currently active malignancy if they have completed therapy and are free of disease for ≥1 year); (14) recent infection requiring systemic anti-infective treatment that was completed ≤14 days prior to enrollment (with the exception of uncomplicated urinary tract infection or upper respiratory tract infection); (15) uncontrolled diabetes mellitus, as defined by HbA1c >8% despite adequate therapy; (16) myocardial infarction during the 6 months prior to enrollment; (17) symptomatic congestive heart failure (CHF), unstable angina pectoris, cardiac arrhythmia, uncontrolled hypertension, or unstable coronary artery disease; (18) history of interstitial lung disease and/or pneumonitis, or pulmonary hypertension; (19) use of strong inhibitors and/or inducers of cytochrome P450 (CYP) 3A4 (CYP3A4) and/or p-glycoprotein (p-GP) within the 14 days prior to receiving the first dose of nab-sirolimus (additionally, use of any known CYP3A4 substrates with a narrow therapeutic window, such as fentanyl, alfentanil, astemizole, cisapride, dihydroergotamine, pimozide, quinidine, terfenadine, within the 14 days prior to receiving the first dose of nab-sirolimus); (20) planned vaccination with live vaccines during treatment with nab-sirolimus, and for 4 weeks after receipt of the last dose of nab-sirolimus (live vaccines may include, but are not limited to, measles, mumps, rubella, oral polio. BCG (Bacillus Calmette-Guerin), yellow fever, varicella, and T21a typhoid); (21) known human immunodeficiency virus (HIV), active hepatitis B or hepatitis C infection(s); (22) active participation in an investigational drug trial for mitochondrial disease within 30 days prior to enrollment (or within 90 days for a trial with an investigational biologic), or disease-related surgical intervention within 30 days prior to enrollment; or (23) any condition (e.g., known or suspected poor compliance, psychological instability, and geographical location) that, in the opinion of the Investigator, may affect the patient's ability to fully comply with all requirements of the study.

The End of Study (EOS) is defined as (1) either the date of the last visit of the last patient to complete the study or (2) the date of collection of the last data point from the last patient that is required for primary, secondary, and/or exploratory analyses, as pre-specified in the protocol.

End of Treatment (EOT) for a patient is defined as the date of the last dose of nab-sirolimus. The EOT assessment for a patient is when safety assessments and procedures are performed after the last treatment, which must occur at least 4 weeks (±7 days) after the last dose of nab-sirolimus.

Follow-up period is the on-study time period after the EOT Visit. All patients that discontinue nab-sirolimus and have not withdrawn full consent to participate in the study continue in the follow-up phase for collection of survival data and clinical activity of nab-sirolimus. Follow up continues approximately every 12 weeks (+/−3 weeks), until death, withdrawal of consent, or the study closes, whichever is the earliest. This evaluation may be made by record review and/or telephone contact.

Two dose reduction levels are permitted. The first dose reduction level is 25% of the total starting dose and the second dose reduction level is 50% of the total starting dose. For example, if a patient is administered 30 mg/m², the two-step dose reduction is an initial dose reduction to 22.5 mg/m² and then, if unacceptable toxicity persists, a second dose reduction to 15 mg/m².

If unacceptable toxicity (≥Grade 3, per CTCAE criteria) occurs, the total starting dose is reduced by 25%. Following the initial dose reduction, if toxicity improves to <Grade 3 within 2 weeks of the initial dose reduction, the patient continues to receive this reduced dose unless further toxicity develops. If toxicity does not improve to <Grade 3 within 2 weeks of the first dose reduction, the total starting dose is reduced by 50%. If toxicity worsens to >Grade 3 within 1 week of the first dose reduction, the total starting dose is reduced by 50%. Following the second dose reduction, if toxicity does improve within 1 week or resolve to an acceptable level (as determined and documented by the investigator), administration of nab-sirolimus is terminated. If, following dose reduction, toxicity has improved to an acceptable level (as determined and documented by the Investigator), patients continue to receive treatment until (1) there is new or recurrent unacceptable toxicity or disease progression, (2) in the opinion of the Investigator, the patient is no longer benefiting from therapy. (3) at the Sponsor's request, or (4) at the discretion of the patient.

Primary endpoints for this study include safety and tolerability. Safety evaluations include a determination of, for example, serious adverse events (SAEs)/adverse events (AEs), laboratory parameter assessments, physical examinations, vital signs, and ECGs.

Secondary endpoints for the study include, but are not limited to: (1) clinical activity, as determined by the change in baseline at 26 weeks in: (a) the Newcastle Pediatric Mitochondrial Disease Scale (NPMDS), (b) Gross Motor Function Measure (GMFM). (c) Quality of Life (PedsQL); (d) Neuromuscular function (Barry Albright Dystonia Scale); and (e) an exploratory assessment of clinical activity, as determined by a non-validated Parent/Direct Caregiver Observer-Reported Outcome Measure in Patients with Leigh syndrome (Obs-RO-Ls); (2) change in respiratory function, as determined by oxygen (O₂) saturation and need for tracheostomy; (3) change in cardiac function; (4) disease progressions as determined by brain MRI; (5) Mental fatigue and physical fatigue (e.g., exercise tolerance); (6) morbidity (overall patient survival); (7) mortality (number of hospitalizations); and (8) pharmacokinetic/pharmacodynamic relationships for select safety and/or clinical endpoints may also be evaluated. Other possible secondary endpoints include Developmental Quotient (minimum 2 years follow-up) and head circumference over time (minimum 2 years follow-up).

Exploratory objectives for the study include, but are not limited to: (1) determination in whole blood (e.g., plasma) of: (a) lactate, (b) ketones (e.g., acetoacetate and β-hydroxybutyrate); and/or (c) metabolites of the glycolytic pathway (e.g., phosphofructokinase, hexokinase, pyruvate kinase, and glucose transporters GLUT 1 to GLUT 5); (2) determination in lymphocytes isolated from whole blood of: total, reduced, and oxidized glutathione peroxidase enzyme activities; (3) determination in leukocytes isolated from whole blood of: pyruvate dehydrogenase enzyme activity; (4) determination in cerebrospinal fluid (CSF) of lactate; (5) determination in purified mitochondria isolated from small muscle biopsy (where feasible) of cytochrome C oxidase activity; (6) respiratory chain enzyme activity: and (7) characterization of potential genetic markers in skin biopsy/fibroblasts that may assist with the identification of future clinical study patients who may benefit from treatment with nab-sirolimus. Other exploratory objectives include determination in patient samples of additional analytes and pharmacokinetics/pharmacodynamics (PK/PD) for select biomarkers.

All patients who receive at least one dose of nab-sirolimus are evaluated for safety. Safety outcomes include serious adverse events (SAEs), adverse events (AEs), laboratory tests (e.g., biochemistry and hematology), vital signs (e.g., blood pressure, pulse, respiration rate, and temperature). ECGs, and incidence of patients experiencing dose modifications, dose delay/dose not given, dose interruptions, and/or premature discontinuation of study drug due to an AE. All AEs are recorded by the investigator from the time the patient signs informed consent until 28 days after the last dose of nab-sirolimus. Adverse events are graded by National Cancer Institute (NCI) Common Terminology Criteria for Adverse Events (CTCAE) and coded using the Medical Dictionary for Medical Activities (MedDRA) and grouped by their system organ class and preferred term. All SAEs (regardless of relationship to IP) are followed until resolution. Summary tables are prepared including the number and percentage of patients with AEs, serious AEs, fatal AEs and other AEs of special interest. An independent Data Monitoring Committee (DMC) assesses safety data (e.g., SAEs, AEs, and selected laboratory parameters) approximately every 6 months after the first 4 patients have been enrolled and treated with at least 2 doses of therapy. The DMC also reviews primary efficacy data for all patients but does not make recommendations regarding the course and/or duration of treatment.

After disease progression has been demonstrated, patients are followed for survival every 12 weeks, or more frequently as needed, until death, withdrawal of consent, or the study closes, whichever is earliest.

Whole blood samples (minimum of 2 mL) for the determination of rapamycin concentrations are collected in Vacutainer® tubes containing potassium-EDTA. Samples are stored frozen at temperatures between −20° C. and −80° C. until shipment for analysis at the central laboratory to be designated by the Sponsor. The whole blood samples are analyzed for total (free+bound) rapamycin using high-performance liquid chromatography-tandem mass spectrometry (HPLC-MS-MS).

Samples are collected immediately pre-dose on Treatment Day 1 and again on Days 8 and 15 (Time 0: before infusion of ABI-009), during infusion (Times: 15 minutes and 30 minutes just before the end of the infusion), and post-infusion (Times: 1.0, 1.5, 2, 4, 6, 8, 24, 48, 72, 96, and 168 hours after completion of the infusion). The concentration-versus-time data for rapamycin in whole blood is analyzed using a noncompartmental analysis technique and WinNonlin software. Calculated parameters include peak concentration (Cₘₐₓ), half-life (t_{1/2}), area under the concentration-time curve (AUC), clearance (CL), and steady-state volume of distribution (Vₛₛ). A simple regression model is applied to assess the relationship of the pharmacokinetic parameters with dose. Pharmacokinetic/pharmacodynamics relationships for the safety and clinical activity endpoints, as well as for select secondary and/or exploratory endpoints, may also be evaluated.

Descriptive statistics are performed on data collected from all patients enrolled in the study. Changes in outcomes measures are calculated individually for each patient and overall mean changes from baseline are analyzed using a Wilcoxon signed rank test. Because age-specific versions of the NPMDS (0-24 months, 2-11 years, and 12-17 years of age at the time of entry) are used in this study, the Wilcoxon test is used to determine overall significance of the treatment effect across the entire patient cohort. Mean changes in treatment effect are also calculated separately for each age group. Statistical significance is defined as p<0.05. Based on the individual primary clinical activity endpoints of NPMDS. GMFM, PEDsQL, and Parent/Direct Caregiver Observer-Reported Outcome Measure in Patients with Leigh's syndrome (Obs-RO-Ls), each patient's overall outcome is categorized as improved, stable, progressing, or death.

Patients without a valid clinical activity (e.g., response) assessment are assigned a best overall response of not evaluable (NE). NE patients are included in the calculation of response rate, and are considered as non-responders. Data from patients who are lost to follow-up or who have missing information before reaching an endpoint in any of the time-to-event analyses are treated as censored, with specific rules to identify the date of censoring.

### Example 5

### Inhibition of Glucose Consumption in IMR90 Fibroblasts

This example demonstrates mTOR inhibition in IMR90 following administration of nab-sirolimus (also referred to as ABI-009), rapamycin, and torin 1.

Human IMR90 fibroblasts (ATCC® CCL-186™; organism: human; cell type: fibroblast; tissue: lung; disease: normal) were administered varying doses of allosteric mTOR inhibitors nab-sirolimus, rapamycin, and torin. Percentage of maximum response of inhibition of glucose usage was measured following administration.

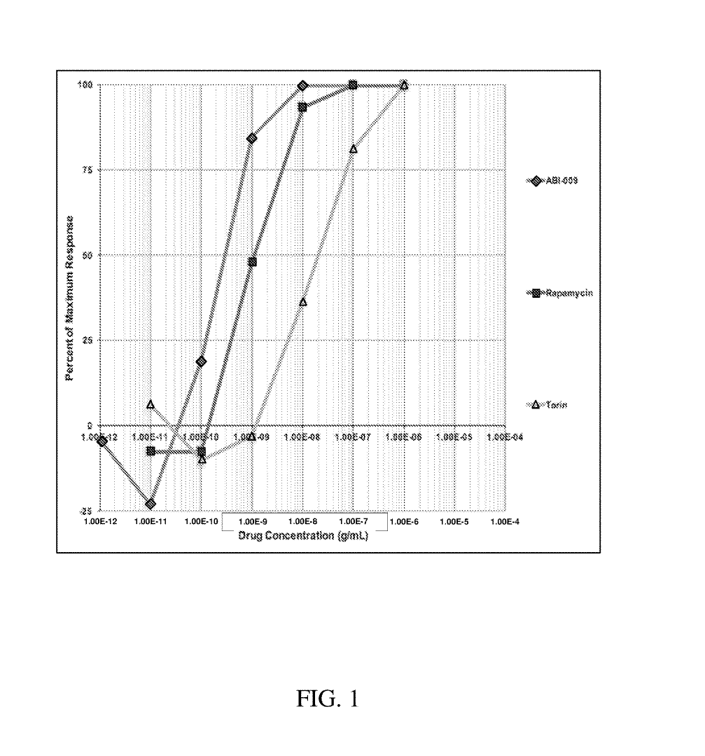

FIG. 1 shows results from the dose-response experiments. For this phenotypic marker of mTOR inhibition, inhibition of glucose usage, nab-sirolimus was at maximal effect at a dose 10× lower than the dose of rapamycin and 100× lower than torin 1. Thus, nab-sirolimus was more potent than either of rapamycin and torin. There was no toxicity observed, even at extremely high doses of nab-sirolimus.

Nab-sirolimus was more potent than either rapamycin or torin 1 in regards to EC50, which was approximately 5 fold lower than regular rapamycin and approximately 100 fold lower than torin 1.

This glucose usage phenotype is fully mTOR dependent and therefore can be used as a direct readout of mTOR inhibition in both primary and immortalized fibroblasts.

### Example 6

### Response on pS6 and Total S6 in IMR90 Following Administration of Nab-Sirolimus and Rapamycin

This example demonstrates the inhibition of S6 phosphorylation in IMR90 fibroblasts following administration of nab-sirolimus (also referred to as ABI-009) and rapamycin.

IMR90 fibroblasts were administered varying doses of nab-sirolimus or rapamycin. Cell lysates were used to determine inhibition of S6 phosphorylation. Presence of vimentin. S6, and phosphorylated S6 (pS6) was measured.

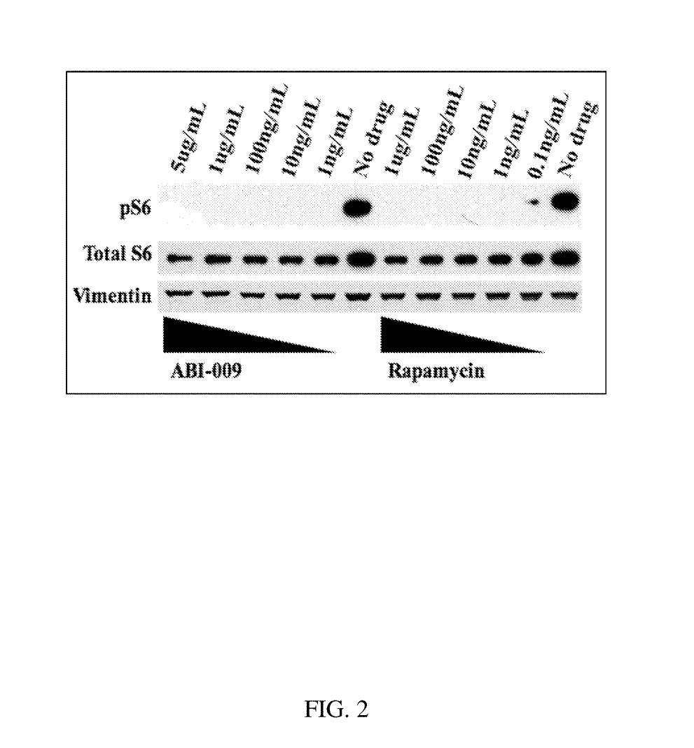

Both nab-sirolimus and rapamycin inhibited 100% of pS6, at the dose of 1 ng/mL (FIG. 2). A slight response curve in total S6 was observed (FIG. 2).

What is claimed is:

1. A method of treating an individual having a mitochondrial-associated disorder comprising administering to the individual an effective amount of an allosteric mTOR inhibitor.

2. The method of claim 1, wherein the individual having a mitochondrial-associated disorder has one or more of the following: an ataxia, a kidney disorder, a liver disorder, a metabolic disorder, a myopathy, a neuropathy, a myelopathy, an encephalopathy, or an oxidative phosphorylation disorder.

3. The method of claim 1 or 2, wherein the individual having a mitochondrial-associated disorder has Leigh syndrome.

4. The method of claim 3, wherein Leigh syndrome is maternally inherited Leigh syndrome.

5. The method of claim 3 or 4, wherein Leigh syndrome is infantile onset Leigh syndrome, juvenile onset Leigh syndrome, or adult onset Leigh syndrome.

6. The method of claim 1 or 2, wherein the individual having a mitochondrial-associated disorder has MELAS syndrome.

7. The method of claim 1 or 2, wherein the individual having a mitochondrial-associated disorder has NARP syndrome.

8. The method of claim 1, wherein the individual having a mitochondrial-associated disorder has one or more of the following: an aging disorder, an autism spectrum disorder, a chronic inflammatory disorder, diabetes mellitus, or a fatty acid oxidation disorder.

9. The method of any one of claims 1-8, wherein the individual having a mitochondrial-associated disorder has a mitochondrial DNA mutation-associated disorder.

10. The method of any one of claims 1-9, wherein the individual having a mitochondrial-associated disorder has a nuclear DNA mutation-associated disorder.

11. The method of any one of claims 1-10, wherein the individual having a mitochondrial-associated disorder has an X chromosome mutation-associated disorder.

12. The method of any one of claims 1-11, wherein the individual is about one month old to about thirty years old.

13. The method of any one of claims 1-12, wherein the age of onset of one or more mitochondrial-associated disorder symptoms in the individual is between about three months old and about two years old.

14. The method of any one of claims 1-13, wherein the individual is a male.

15. The method of any one of claims 1-14, wherein the individual has a mutation in one or more of the following genes: LRPPRC, MT-ATP6, MT-ND1, MT-ND2, MT-ND3, MT-ND5, MT-ND6, MT-TL1, MT-TH, MT-TV, NDUFS1, NDUFS2, NDUFS3, NDUFS4, NDUFS7, NDUFS8, or SURF1.

16. The method of any one of claims 1-15, wherein the individual is selected for treatment based on the ratio of lactate to pyruvate in their blood, plasma, cerebrospinal fluid, or urine.

17. The method of claim 16, wherein the ratio of lactate to pyruvate is at least 10:1.

18. The method of claim 16 or 17, wherein the ratio of lactate to pyruvate is at least 20:1.

19. A method of inhibiting cellular glucose consumption in an individual comprising administering to the individual an effective amount of an allosteric mTOR inhibitor.

20. A method of treating an individual having a metabolic disorder comprising administering to the individual an effective amount of an allosteric mTOR inhibitor.

21. A method of treating an individual having a disease comprising administering to the individual an effective amount of an allosteric mTOR inhibitor, wherein the disease is selected from the group consisting of fetal dilated cardiomyopathy, tuberous sclerosis complex (TSC) and related disorders, childhood onset cardiomyopathy, Noonan syndrome, polycystic kidney disease, age-related and genetically induced hypertrophic cardiomyopathy, and a rheumatic disease.

22. The method of any one of claims 1-21, wherein the allosteric mTOR inhibitor is in a composition comprising nanoparticles comprising the allosteric mTOR inhibitor and an albumin.

23. The method of any one of claims 1-22, wherein the allosteric mTOR inhibitor is a limus drug.

24. The method of claim 23, wherein the limus drug is sirolimus.

25. The method of any one of claims 1-24, wherein the effective amount of allosteric mTOR inhibitor is about 1 mg/m² to about 150 mg/m².

26. The method of any one of claims 1-25, wherein the effective amount of allosteric mTOR inhibitor is administered weekly.

27. The method of any one of claims 1-25, wherein the effective amount of allosteric mTOR inhibitor is administered once every two weeks.

28. The method of any one of claims 1-25, wherein the effective amount of allosteric mTOR inhibitor is administered daily.

29. The method of any one of claims 1-25, wherein the effective amount of allosteric mTOR inhibitor is administered once every three days.

30. The method of any one of claims 1-29, wherein the effective amount of allosteric mTOR inhibitor is administered intravenously, intraarterially, intraperitoneally, intravesicularly, subcutaneously, intrathecally, intrapulmonarily, intramuscularly, intratracheally, intraocularly, transdermally, intradermally, orally, intraportally, intrahepatically, by hepatic arterial infusion, or by inhalation.

31. The method of claim 30, wherein the effective amount of allosteric mTOR inhibitor is administered intravenously.

32. The method of any one of claims 22-31, wherein the nanoparticles in the composition have an average diameter of no greater than about 150 nm.

33. The method of claim 32, wherein the nanoparticles in the composition have an average diameter of no greater than about 120 nm.

34. The method of any one of claims 22-33, wherein the allosteric mTOR inhibitor in the nanoparticles is associated with the albumin.

35. The method of any one of claims 22-34, wherein the weight ratio of albumin and allosteric mTOR inhibitor in the nanoparticle composition is about 1:1 to about 9:1.

36. The method of claim 35, wherein the weight ratio of albumin and allosteric mTOR inhibitor in the nanoparticle composition is about 8:1, about 8.5:1, or about 9:1.

37. The method of any one of claims 22-36, wherein the albumin is human albumin.

38. The method of any one of claims 22-36, wherein the albumin is human serum albumin.

39. The method of any one of claims 1-38, wherein the individual is human.

40. The method of any one of claims 1-39, wherein the individual has not been previously treated with an allosteric mTOR inhibitor.
